(19) United States
(12) Patent Application Publication
(10) Pub. No.: US 2023/0364020 A1
(43) Pub. Date: Nov. 16, 2023

(54) HYDROXYPROPYL METHYL CELLULOSE DERIVATIVES TO STABILIZE POLYPEPTIDES

(71) Applicant: Genentech, Inc., South San Francisco, CA (US)

(72) Inventors: Purnendu Kumar NAYAK, South San Francisco, CA (US); Debby Pei-Shan CHANG, South San Francisco, CA (US); Karthikan RAJAGOPAL, South San Francisco, CA (US); Jonathan ZARZAR, Pacifica, CA (US)

(21) Appl. No.: 18/194,542

(22) Filed: Mar. 31, 2023

Related U.S. Application Data
(60) Provisional application No. 63/326,772, filed on Apr. 1, 2022.

(51) Int. Cl. A61K 9/16; C07K 16/32; C07K 16/22; C07K 16/42

(52) U.S. Cl. CPC A61K 9/1652; A61K 9/1617; A61K 9/1623; A61K 9/1647; C07K 16/32; C07K 16/22; C07K 16/4291; A61K 9/1694

(57) ABSTRACT

The present invention provides formulations comprising polypeptides and hydroxypropyl methylcellulose acetate succinate (HPMCAS) derivatives. The formulations are stable; for example, during high temperature processing and in possible low pH environments. In addition, the HPMCAS derivatives provide protection to a pH sensitive protein against acidic degradation products from aqueous hydrolysis of poly(lactic-co-glycolic acid) (PLGA) in PLGA-based delivery systems.

### CROSS-REFERENCE TO RELATED APPLICATIONS

This application claims priority under 35 U.S.C. Section 119(e) and the benefit of U.S. Provisional Patent Application No. 63/326,772, filed Apr. 1, 2022, which is incorporated herein by reference in its entirety.

### FIELD OF THE INVENTION

The present invention provides the use of hydroxypropyl methylcellulose acetate succinate (HPMCAS) derivatives as polymeric excipients for stabilizing polypeptides; for example, during high temperature processing and in possible low pH environments. In addition, the HPMCAS derivatives provide protection to a pH sensitive protein against acidic degradation products from aqueous hydrolysis of poly(lactic-co-glycolic acid) (PLGA) in PLGA-based delivery systems.

### BACKGROUND OF THE INVENTION

Protein-based therapeutics are susceptible to physical and chemical degradation reactions during manufacture, storage and delivery (Le Basle, Y et al., J Pharm Sci, 2020. 109(1): p. 169-190). While an understanding of how excipients stabilize proteins in liquid and solid state has enabled the successful development of many protein-based drug products there is still a need to identify new excipients and design formulations to enable the development of polymer-based sustained release (Fu, K et al., Nat Biotechnol, 2000, 18(1):24-5; Putney, SD and PA Burke, Nat Biotechnol, 1998. 16(2):153-7). Protein stability encompasses physicochemical phenomena and changes in conformational structures that are not relevant for small molecule therapeutics (Manning, M C et al., Pharm Res, 2010, 27(4):544-75). In case of a polymeric controlled release system the protein drug should remain within the formulation at physiological temperatures for few weeks to a year (Zhu, G et al., Nat Biotechnol, 2000, 18(1): 52-7; Vaishya, R et al., Expert Opin Drug Deliv, 2015, 12(3): 415-40. For example, in the preparation of drug-loaded PLGA rods the drug is exposed to a high temperature during manufacturing and later on to a low pH microenvironment due to polymer hydrolysis when the rod resides at body temperature for an extended duration. Without stabilizing excipients the protein drug is subject to possible degradation both inside the delivery system and upon its release in vivo (Rajagopal, K et al., J Pharm Sci, 2013, 102(8):2655-66).

The preparation of PLGA rods using a hot melt extrusion process involves mixing of a spray dried protein formulation with solid PLGA, micro-compounding at 90° C. followed by extrusion, cooling and cutting of the extrudate to solid rods to desired size (Rajagopal, K et al., J Pharm Sci, 2013, 102(8):2655-66). It was demonstrated previously that inclusion of hydrophilic small molecule excipients such as trehalose or ectoine in spray dried formulations stabilize proteins at elevated temperature such that a hot-melt extrusion process can be used for the preparation of protein-loaded PLGA rods (Rajagopal, K et al., Mol Pharm, 2019. 16(1):349-358; Nayak, P K et al., Molecular Pharmaceutics, 2020, 17(9): 3291-3297. While these excipients stabilize proteins in solid state during the preparation and shelf-life storage of PLGA rods, they do not provide stability against low pH micro-environment created due to the hydrolytic polymer degradation after the rods are implanted in the body (Chang, D P et al., J Pharm Sci, 2015, 104(10):3404-17). Since the hydrolysis of polymer results in the formation of lactic and glycolic acids (Pitt, C G et al., Biomaterials, 1981, 2(4):215-20), the accumulation of those acids within a PLGA delivery system creates an acidic microenvironment (Shenderova, A et al., Pharm Res, 1999. 16(2): 241-8; Brunner, A et al., Pharm Res, 1999, 16(6): 847-53. Not only do these excipients lack buffer capacity to neutralize the acidic micro-environment they are also released faster than the protein due to their small size and hydrophilicity. This scenario leaves the protein unprotected within the rod and contributes to aggregation, incomplete drug release and facilitates low-pH chemical degradation reactions such as isomerization and fragmentation potentially leading to a loss in activity (Rajagopal, K et al., Mol Pharm, 2019. 16(1):349-358; Liu, Y and SP Schwendeman, Mol Pharm, 2012, 9(5): 1342-50; Fu, K et al., Pharm Res, 2000, 17(1):100-6; Lu, W and T G Park, PDA J Pharm Sci Technol, 1995, 49(1): 13-9; Shao, P G. and L C Bailey, Pharm Dev Technol, 2000, 5(1):1-9). Studies from Zhu et al. have shown that large globular proteins like BSA undergo widespread aggregation inside PLGA system during prolong incubation at 37° C. in PBS buffer (Zhu, G and SP Schwendeman, Pharm Res, 2000, 17(3): 351-7). Therefore, excipients that neutralize the acidic microclimate and counter protein degradation are necessary for stable protein release from PLGA systems.

Various routes of protein stability in PLGA devices have been explored such as addition of basic additives like Mg(OH)₂ to stabilize BSA (Thu, G et al., Nat Biotechnol, 2000, 18(1): 52-7) and divalent metal cation salts like CaCl₂) or MnCl₂ to stabilize octreotide (Sophocleous, A M et al., J Control Release, 2009. 137(3): 179-84). However, the above approaches rely on small inorganic ions as additives to either neutralize the degradant products or prevent protein interactions with the degradant species from PLGA hydrolysis, without providing protection as excipients to protein.

What is needed is the use of polymeric excipients for stabilizing proteins in spray dried formulations at elevated temperature and also to neutralize the low pH environment created within PLGA.

All references cited herein, including patent applications and publications, are incorporated by reference in their entirety.

### BRIEF SUMMARY

In some aspects, the invention provides a formulation comprising a polypeptide and a hydroxypropyl methylcellulose acetate succinate (HPMCAS). In some embodiments, the HPMCAS comprises from about 8% to about 12% acetate and from about 7% to about 15% succinate. In some embodiments, the HPMCAS comprises about 8% acetate and about 15% succinate (HPMCAS-LF). In other embodiments, the HPMCAS comprises about 12% acetate and about 7% succinate (HPMCAS-HF). In some embodiments, the ratio of the HPMCAS to protein in the formulation is about 10:1 (mg/mg) to about 1:10 (mg/mg). In some embodiments, the ratio of the HPMCAS to protein in the formulation is from about 4:1 (mg/mg) to about 1:4 (mg/mg). In some embodiments, the ratio of the HPMCAS to protein in the formulation is about 4:1 (mg/mg). In some embodiments, the ratio of the HPMCAS to protein in the formulation is about 1:1 (mg/mg). In some embodiments, the ratio of the HPMCAS to protein in the formulation is about 1:4 (mg/mg). In some embodiments, the ratio of the HPMCAS to protein in the formulation is about 1:1 (mg/mg) to about 1:5 (mg/mg). In some embodiments, the formulation further comprises a histidine buffer. In some embodiments, the histidine buffer is a histidine HCl buffer. In some embodiments, the histidine buffer is in the formulation at a concentration of about 5 mM to about 20 mM. In some embodiments, the histidine buffer is in the formulation at a concentration of about 10 mM. In some embodiments, the formulation further comprises a polysorbate. In some embodiments, the polysorbate is polysorbate 20. In some embodiments, the polysorbate in the formulation at a concentration of about 0.005% (w/v) to about 0.5% (w/v). In some embodiments, the polysorbate is in the formulation at a concentration of about 0.01% (w/v). In some embodiments, the pH of the formulation is about 5.5 to about 7.0. In some embodiments, the pH of the formulation is about 6.5.

In some embodiments if the invention, the polypeptide in the formulation is an antibody. In some embodiments, the polypeptide is a monoclonal antibody. In some embodiments, the polypeptide is a human antibody, a chimeric antibody or a humanized antibody. In some embodiments, the antibody is an antibody fragment selected from a Fab, Fab′-SH, Fv, scFv, and (Fab′)2 fragment. In some embodiments, the antibody fragment is a (Fab′)2 fragment. In some embodiments, the polypeptide is in the formulation at a concentration of about 5 mg/mL to about 100 mg/mL, or about 10 mg/mL to about 80 mg/mL In some embodiments, the polypeptide is in the formulation at a concentration of about 10 mg/mL.

In some aspects, the invention provides a formulation comprising an antibody at a concentration of about 5 mg/mL to about 50 mg/mL, an HPMCAS-LF, wherein the ratio of the HPMCAS-LF to antibody in the formulation is about 1:1 (mg/mg), a histidine-HCl buffer at a concentration of about 5 mM to about 20 mM, polysorbate 20 at a concentration of about 0.005% (w/v) to about 0.5% (w/v), and wherein the pH of the formulation is about 5.5 to about 7.0. In some aspects, the invention provides a formulation comprising an antibody at a concentration of about 10 mg/mL, an HPMCAS-LF, wherein the ratio of the HPMCAS-LF to antibody in the formulation is about 1:1 (mg/mg), a histidine-HCl buffer at a concentration of about 10 mM, polysorbate 20 at a concentration of about 0.01% (w/v), and wherein the pH of the formulation is about 6.5. In some aspects, the invention provides a formulation comprising an antibody at a concentration of about 5 mg/mL to about 50 mg/mL, an HPMCAS-HF, wherein the ratio of the HPMCAS-HF to antibody in the formulation is about 1:1 (mg/mg), a histidine-HCl buffer at a concentration of about 5 mM to about 20 mM, polysorbate 20 at a concentration of about 0.005% (w/v) to about 0.5% (w/v), and wherein the pH of the formulation is about 5.5 to about 7.0. In some aspects, the invention provides a formulation comprising an antibody at a concentration of about 10 mg/mL, an HPMCAS-HF, wherein the ratio of the HPMCAS-HF to antibody in the formulation is about 1:1 (mg/mg), a histidine-HCl buffer at a concentration of about 10 mM, polysorbate 20 at a concentration of about 0.01% (w/v), and wherein the pH of the formulation is about 6.5.

In some embodiments of the inventions, the formulation is spray dried to form a spray dried formulation of a polypeptide or antibody. In some embodiments, the polypeptide (e.g, an antibody) in the formulation is stable at about 90° C. for at least about 5 hours and/or is stable at about 37° C. for at least about 4 weeks. In some embodiments, the polypeptide in the formulation shows reduced aggregation and/or reduced chemical degradation. In some embodiments, the reduced chemical degradation comprises reduced formation of succinimide variants of the polypeptide and/or reduced formation of pyroglutamate variants of the polypeptide. In some embodiments, the formulation is an amorphous glassy formulation.

In some embodiments of the invention, the spray dried formulation of a polypeptide (e.g., an antibody) is encapsulated in a polymer system that produces acidic microclimate. In some embodiments, the spray dried formulation of a polypeptide is encapsulated in lactic acid/glycolic acid polymer system. In some embodiments, the spray dried formulation of a polypeptide is encapsulated in poly(lactic-co-glycolic acid) (PLGA). In some embodiments, the spray dried formulation of a polypeptide is encapsulated in a PLGA rod.

In some aspects, the invention provides a composition comprising any of the formulations described herein; for example, a composition comprising a spray dried formulation of a polypeptide (e.g., an antibody) as described herein. In some embodiments, the invention provides a composition comprising a lactic acid/glycolic acid polymer particle comprising any of the formulations described herein. In some embodiments, the invention provides a composition comprising a lactic acid/glycolic acid polymer particle comprising any of the spray dried formulations described herein. In some embodiments, the lactic acid/glycolic acid polymer particle is a PLGA particle. In some embodiments, the lactic acid/glycolic acid polymer particle is a PLGA rod.

In some aspects, the invention provides methods of preparing a spray dried polypeptide, the method comprising preparing a formulation as described herein, and subjecting the formulation to spray drying. In some aspects, the invention provides methods of preparing a spray dried antibody, the method comprising preparing a formulation as described herein, and subjecting the formulation to spray drying. In some embodiments, the spray drying is performed using a spray dryer comprising an inlet and an outlet. In some embodiments, the inlet has a temperature of about 90° C. to about 120° C., about 100° C., or about 110° C. In some embodiments, the outlet has a temperature of about 60° C.

In some aspects, the invention provides methods of preparing a lactic acid/glycolic acid polymer particle comprising a polypeptide, the method comprising encapsulating the formulation as described herein in a lactic acid/glycolic acid polymer system. In some aspects, the invention provides methods of preparing a lactic acid/glycolic acid polymer particle comprising a polypeptide, the method comprising encapsulating the spray dried formulation as described herein in a lactic acid/glycolic acid polymer system. In some embodiments, the lactic acid/glycolic acid polymer particle is a PLGA particle.

In some aspects, the invention provides articles of manufacture comprising any of the formulations (e.g., spray dried formulations) or compositions as described herein.

### BRIEF DESCRIPTION OF THE DRAWINGS

FIG. 1 shows the structures of trehalose and a generic structure of cellulose. The bottom panel shows the nature of R groups that define Metolose, AS-LF and AS-HF and the molecular weight and glass transition temperature of polymeric excipients along with Trehalose

FIG. 2 shows the formulation glass transition temperature as represented by differential scanning calorimetry (DSC) thermograms of all five, spray dried Fab2 formulations

FIG. 3 is a schematic showing the preparation of spray dried AS-LF and AS-HF formulations. AS-HF or AS-LF polymers were dissolved in water, titrated with histidine base to pH 6.5 and then mixed with Fab2 solution in pH 5.5 buffer. The cloudy polymer and protein mixed solution was spray dried to yield a solid formulation.

FIGS. 4A and 4B show characterization of spray dried formulations. FIG. 4A shows size distribution of spray dried Fab2 formulation measured using laser diffraction. FIG. 4B shows SEM images of spray dried formulations. Scale bar in each image corresponds to 10 μm.

FIGS. 5A and 5B show Fab2 aggregation in spray dried formulations. The change in monomer content as a function of time for Fab2 in spray dried formulations after exposure to 90° C. for 5 hours (FIG. 5A) and 37° C. for 4 weeks (FIG. 5B).

FIGS. 6A-6D show chemical degradation of Fab2 in spray dried formulations. Ion-exchange chromatogram of Fab2 in different formulations after 5 hours at 90° C. and (FIG. 6A) 4 weeks at 37° C. (FIG. 6B). Pyroglutamate formation, measured as ratio of PyroE and main peak areas, with time at 90° C. (FIG. 6C) and 37° C. (FIG. 6D).

FIGS. 7A and 7B show the buffer capacity of AS excipients. HPMCAS excipients resist change in pH (FIG. 7A) upon titration with a strong base (0.5 N NaOH) in raw material form (FIG. 7B) and during titration with a strong acid (0.1 N HCl) in spray dried Fab2 formulations.

FIG. 8 shows Fab2 stability within the PLGA rod. The change in extracted Fab2 monomer content upon recovery after 3 weeks within a PLGA rod at 37° C. for all four formulations.

FIG. 9 is a schematic showing protein and polymer distribution in spray dried formulation. Spray drying a protein-polymer mixture produces two types of distribution in solid state. Electrostatic interaction between Fab2 and HPMCAS polymers ensures uniform blending in solid state, whereas in metolose formulation physico-chemical incompatibility causes demixing and phase separation after spray drying.

FIG. 10 shows excipient glass transition temperature as represented by differential scanning calorimetry (DSC) thermograms of all four excipients used in our study.

FIG. 11 shows MAb1 aggregation in spray dried formulations. The change in monomer content as a function of time is shown for MAb1 in spray dried formulations containing different excipient to protein weight ratios (E/P) after exposure to 90° C. for various periods of time (0 to 5 hours).

FIG. 12 shows MAb1 aggregation in spray dried formulations. The change in monomer content as a function of time is shown for MAb1 in spray dried formulations containing different excipient to protein weight ratios (E/P) after exposure to 37° C. for various periods of time (0 to 8 weeks).

FIG. 13 shows MAb2 aggregation in spray dried formulations. The change in monomer content as a function of time is shown for MAb2 in spray dried formulations containing different excipient to protein weight ratios (E/P) after exposure to 90° C. for various periods of time (0 to 4 or 5 hours).

FIG. 14 shows MAb2 aggregation in spray dried formulations. The change in monomer content as a function of time is shown for MAb2 in spray dried formulations containing different excipient to protein weight ratios (E/P) after exposure to 37° C. for various periods of time (0-8 weeks).

FIG. 15 shows buffer capacity of MAb1 formulations containing different excipients. Change in pH is shown upon addition of dilute hydrochloric acid to MAb1 formulations dissolved in water after spray drying. The arrow indicates increase in excipient content in formulations with protein.

FIG. 16 shows buffer capacity of MAb2 formulations containing different excipients. Change in pH is shown upon addition of dilute hydrochloric acid to MAb2 formulations dissolved in water after spray drying. The arrow indicates increase in excipient content in formulations with protein.

FIG. 17 shows MAb1 stability in PLGA rods. The change in extracted MAb1 monomer content upon recovery after 4 weeks within PLGA rods at 37° C. is shown for four formulations.

FIG. 18 shows MAb2 stability in PLGA rods. The change in extracted MAb2 monomer content upon recovery after 4 weeks within PLGA rods at 37° C. is shown for four formulations.

FIG. 19 shows MAb3 aggregation in spray dried formulations. The change in monomer content as a function of time is shown for MAb3 in spray dried formulations containing different excipient to protein weight ratios (E/P) after exposure to 90° C. for various periods of time (0 to 5 hours).

FIG. 20 shows MAb3 aggregation in spray dried formulations. The change in monomer content as a function of time is shown for MAb3 in spray dried formulations containing different excipient to protein weight ratios (E/P) after exposure to 37° C. for various periods of time (0-3 months).

### DETAILED DESCRIPTION OF THE INVENTION

In some aspects, the invention provides formulations comprising a polypeptide (e.g., an antibody) and a hydroxypropyl methylcellulose acetate succinate (HPMCAS) derivative. The use of HPMCAS provides stability to the protein during high temperature processing and in possible low pH environments of a drug delivery platform. Moreover, inside a poly(lactic-co-glycolic acid) (PLGA) based delivery system HPMCAS provides protection to a pH sensitive polypeptide against acidic degradation products from aqueous hydrolysis of PLGA. In some embodiments, the HPMCAS comprises about 8% acetate and about 15% succinate (HPMCAS-LF) or about 12% acetate and about 7% succinate (HPMCAS-HF).

In some aspects, the invention provides formulations comprising a polypeptide (e.g., an antibody) at a concentration of about 5 mg/mL to about 50 mg/mL, an HPMCAS, wherein the ratio of the HPMCAS to polypeptide in the formulation is about 1:1 (mg:mg), a histidine-HCl buffer at a concentration of about 5 mM to about 20 mM, polysorbate 20 at a concentration of about 0.005% (w/v) to about 0.5% (w/v), and wherein the pH of the formulation is about 6.0 to about 7.0.

In some aspects, the invention provides spray dried formulations comprising an polypeptide (e.g., an antibody) at a concentration of about 5 mg/mL to about 50 mg/mL, an HPMCAS, wherein the ratio of the HPMCAS to polypeptide in the formulation is about 1:1 (mg:mg), a histidine-HCl buffer at a concentration of about 5 mM to about 20 mM, polysorbate 20 at a concentration of about 0.005% (w/v) to about 0.5% (w/v), and wherein the pH of the formulation is about 6.0 to about 7.0, wherein the polypeptide in the formulation is stable at about 90° C. for at least about 5 hours and/or is stable at about 37° C. for at least about 4 weeks.

Methods of preparing the formulations, spray dried formulations, and PLGA-encapsulated polypeptides, as well as articles of manufacture comprising the formulations, spray dried formulations, and PLGA-encapsulated polypeptides are also provided.

### Definitions

“Spray drying” refers to the process of atomizing and drying a liquid or slurry comprising a protein or monoclonal antibody using gas (usually air or nitrogen) at a temperature above ambient temperature so as to produce dry powder particles comprising the protein or monoclonal antibody. During the process, liquid evaporates and dry particles form. In one embodiment, the spray drying is performed using a spray dryer, e.g. which has an air inlet temperature from about 80° C. to about 220° C. and an air outlet temperature from about 50° C. to about 100° C. Particles can be separated from the gas by various methods such as cyclone, high pressure gas, electrostatic charge, etc. This definition of spray drying herein expressly excludes freeze drying or crystallizing the monoclonal antibody.

A “dry” particle, protein, or monoclonal antibody herein has been subjected to a drying process such that its water content has been significantly reduced. In one embodiment, the particle, protein, or monoclonal antibody has a water content of less than about 10%, for example less than about 5%, e.g., where water content is measured by a chemical titration method (e.g. Karl Fischer method) or a weight-loss method (high-temperature heating).

As used herein, a “pre-spray dried preparation” refers to a preparation of a polypeptide (e.g., an antibody) and one or more excipients, such as stabilizers (e.g. an HPMCAS derivative) and, optionally, a buffer. In one embodiment, the preparation is in liquid form.

A “suspension formulation” is a liquid formulation comprising solid particles (e.g. spray dried antibody particles) dispersed throughout a liquid phase in which they are not soluble. In one embodiment, the solid particles in the suspension formulation have an average particle diameter from about 2 to about 30 microns, e.g. from about 5 to about 10 microns (e.g. as analyzed by laser diffraction). Optionally, the solid particles in the suspension formulation have a peak (highest percentage) particle size of less than about 30 micron, and optionally less than about 10 microns (e.g. as analyzed by laser diffraction). The suspension formulation may be prepared by combining spray dried antibody particles with a non-aqueous suspension vehicle. In one embodiment, the suspension formulation is adapted for, or suitable for, subcutaneous administration to a subject or patient.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

“Purified” polypeptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity.

A polypeptide “which binds” an antigen of interest, e.g. a tumor-associated polypeptide antigen target, is one that binds the antigen with sufficient affinity such that the polypeptide is useful as a diagnostic and/or therapeutic agent in targeting a cell or tissue expressing the antigen, and does not significantly cross-react with other polypeptides. In such embodiments, the extent of binding of the polypeptide to a “non-target” polypeptide will be less than about 10% of the binding of the polypeptide to its particular target polypeptide as determined by fluorescence activated cell sorting (FACS) analysis or radioimmunoprecipitation (RIA).

With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term “immunoglobulin” (Ig) is used interchangeable with antibody herein.

Antibodies are naturally occurring immunoglobulin molecules which have varying structures, all based upon the immunoglobulin fold. For example, IgG antibodies have two “heavy” chains and two “light” chains that are disulphide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions (“HVRs”), which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three hypervariable regions, for example, complementarity determining regions (“CDRs”), each of which contains a “hypervariable loop”, and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by V_{H} and V_{L} domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between V_{H} and V_{L}.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

“Framework” or “FR” residues are those variable domain residues other than the hypervariable region residues as herein defined.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), triabody, linear antibodies(e.g., U.S. Pat. No. 5,641,870, Example 2; Zapata et al., Protein Eng. 8(10):1057-1062 (1995)); one-armed antibodies, single variable domain antibodies, minibodies, single-chain antibody molecules; multispecific antibodies formed from antibody fragments (e.g., including but not limited to, db-Fc, taDb-Fc, taDb-CH3, (scFV)4-Fc, sc-Fv, di-scFv, bi-scFv, or tandem (di,tri)-scFv); Bi-specific T-cell engagers (BiTEs), and trifunctional antibody.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “multispecific antibody” is used in the broadest sense and specifically covers an antibody that has polyepitopic specificity. Such multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. “Polyepitopic specificity” refers to the ability to specifically bind to two or more different epitopes on the same or different target(s). “Monospecific” refers to the ability to bind only one epitope. According to one embodiment, the multispecific antibody is an IgG antibody that binds to each epitope with an affinity of 5 M to 0.001 pM, 3 M to 0.001 pM, 1 M to 0.001 pM, 0.5 M to 0.001 pM, or 0.1 M to 0.001 pM.

The expression “single domain antibodies” (sdAbs) or “single variable domain (SVD) antibodies” generally refers to antibodies in which a single variable domain (VH or VL) can confer antigen binding. In other words, the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; FEBs Lett (1994) 339:285-290; WO00/29004; WO 02/051870).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

For the purposes herein, an “intact antibody” is one comprising heavy and light variable domains as well as an Fc region. The constant domains may be native sequence constant domains (e.g. human native sequence constant domains) or amino acid sequence variant thereof. Preferably, the intact antibody has one or more effector functions.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

A “naked antibody” is an antibody (as herein defined) that is not conjugated to a heterologous molecule, such as a cytotoxic moiety or radiolabel.

In some embodiments, antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PB MC) and Natural Killer (NK) cells. Alternatively, or additionally, ADCC activity of the molecule of interest may be assessed in vivo, e.g., in an animal model such as that disclosed in Clynes et al., Proc. Natl. Acad. Sci. (USA) 95:652-656 (1998).

“Human effector cells” are leukocytes that express one or more FcRs and perform effector functions. In some embodiments, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Formulations

In some aspects, the invention provides formulations comprising a polypeptide and a hydroxypropyl methylcellulose acetate succinate (HPMCAS). In some embodiments, the HPMCAS in the formulation comprises between about 1% acetate to about 20% acetate. In some embodiments, the HPMCAS in the formulation comprises any of about 1%, 2%, 3%, 4%, 5%, 6%, 7%, 8%, 9%, 10%, 11%, 12%, 13%, 14%, 15%, 16%, 17%, 18%, 19%, 20%, or greater than 20% acetate. In some embodiments, the HPMCAS in the formulation comprises between about 1% succinate to about 20% succinate. In some embodiments, the HPMCAS in the formulation comprises any of about 1%, 2%, 3%, 4%, 5%, 6%, 7%, 8%, 9%, 10%, 11%, 12%, 13%, 14%, 15%, 16%, 17%, 18%, 19%, 20%, or greater than 20% succinate. In some embodiments, the HPMCAS in the formulation comprises between about 1% acetate to about 20% acetate and between about 1% succinate to about 20% acetate. In some embodiments, the HPMCAS in the formulation comprises about 8% acetate and about 15% succinate (HPMCAS-LF) or about 12% acetate and about 7% succinate (HPMCAS-HF).

In some embodiments, the ratio of the HPMCAS to protein in the formulation is between about 10:1 (mg/mg) to about 1:10 (mg/mg). In some embodiments, the ratio of the HPMCAS to protein in the formulation is between any of about 1:1 (mg/mg) to 1:10 (mg/mg), 1:1 (mg/mg) to 1:9 (mg/mg), 1:1 (mg/mg) to 1:8 (mg/mg), 1:1 (mg/mg) to 1:7 (mg/mg), 1:1 (mg/mg) to 1:6 (mg/mg), 1:1 (mg/mg) to 1:5 (mg/mg), 1:1 (mg/mg) to 1:4 (mg/mg), 1:1 (mg/mg) to 1:3 (mg/mg), 1:1 (mg/mg) to 1:2 (mg/mg), 1:2 (mg/mg) to 1:10 (mg/mg), 1:2 (mg/mg) to 1:9 (mg/mg), 1:2 (mg/mg) to 1:8 (mg/mg), 1:2 (mg/mg) to 1:7 (mg/mg), 1:2 (mg/mg) to 1:6 (mg/mg), 1:2 (mg/mg) to 1:5 (mg/mg), 1:2 (mg/mg) to 1:4 (mg/mg), 1:2 (mg/mg) to 1:3 (mg/mg), 1:3 (mg/mg) to 1:10 (mg/mg), 1:3 (mg/mg) to 1:9 (mg/mg), 1:3 (mg/mg) to 1:8 (mg/mg), 1:3 (mg/mg) to 1:7 (mg/mg), 1:3 (mg/mg) to 1:6 (mg/mg), 1:3 (mg/mg) to 1:5 (mg/mg), 1:3 (mg/mg) to 1:4 (mg/mg), 1:4 (mg/mg) to 1:10 (mg/mg), 1:4 (mg/mg) to 1:9 (mg/mg), 1:4 (mg/mg) to 1:8 (mg/mg), 1:4 (mg/mg) to 1:7 (mg/mg), 1:4 (mg/mg) to 1:6 (mg/mg), 1:4 (mg/mg) to 1:5 (mg/mg), 1:5 (mg/mg) to 1:10 (mg/mg), 1:5 (mg/mg) to 1:9 (mg/mg), 1:5 (mg/mg) to 1:8 (mg/mg), 1:5 (mg/mg) to 1:7 (mg/mg), 1:5 (mg/mg) to 1:6 (mg/mg), 1:6 (mg/mg) to 1:10 (mg/mg), 1:6 (mg/mg) to 1:9 (mg/mg), 1:6 (mg/mg) to 1:8 (mg/mg), 1:6 (mg/mg) to 1:7 (mg/mg), 1:7 (mg/mg) to 1:10 (mg/mg), 1:7 (mg/mg) to 1:9 (mg/mg), 1:7 (mg/mg) to 1:8 (mg/mg), 1:8 (mg/mg) to 1:10 (mg/mg), 1:8 (mg/mg) to 1:9 (mg/mg), or 1:9 (mg/mg) to 1:10 (mg/mg). In some embodiments, the ratio of the HPMCAS to protein in the formulation is between any of about 10:1 (mg/mg) to 1:1 (mg/mg), 9:1 (mg/mg) to 1:1 (mg/mg), 8:1 (mg/mg) to 1:1 (mg/mg), 7:1 (mg/mg) to 1:1 (mg/mg), 6:1 (mg/mg) to 1:1 (mg/mg), 5:1 (mg/mg) to 1:1 (mg/mg), 4:1 (mg/mg) to 1:1 (mg/mg), 3:1 (mg/mg) to 1:1 (mg/mg), 2:1 (mg/mg) to 1:1 (mg/mg), 10:1 (mg/mg) to 2:1 (mg/mg), 9:1 (mg/mg) to 2:1 (mg/mg), 8:1 (mg/mg) to 2:1 (mg/mg), 7:1 (mg/mg) to 2:1 (mg/mg), 6:1 (mg/mg) to 2:1 (mg/mg), 5:1 (mg/mg) to 2:1 (mg/mg), 4:1 (mg/mg) to 2:1 (mg/mg), 3:1 (mg/mg) to 2:1 (mg/mg), 10:1 (mg/mg) to 3:1 (mg/mg), 9:1 (mg/mg) to 3:1 (mg/mg), 8:1 (mg/mg) to 3:1 (mg/mg), 7:1 (mg/mg) to 3:1 (mg/mg), 6:1 (mg/mg) to 3:1 (mg/mg), 5:1 (mg/mg) to 3:1 (mg/mg), 4:1 (mg/mg) to 3:1 (mg/mg), 10:1 (mg/mg) to 4:1 (mg/mg), 9:1 (mg/mg) to 4:1 (mg/mg), 8:1 (mg/mg) to 4:1 (mg/mg), 7:1 (mg/mg) to 4:1 (mg/mg), 6:1 (mg/mg) to 4:1 (mg/mg), 5:1 (mg/mg) to 4:1 (mg/mg), 10:1 (mg/mg) to 5:1 (mg/mg), 9:1 (mg/mg) to 5:1 (mg/mg), 8:1 (mg/mg) to 5:1 (mg/mg), 7:1 (mg/mg) to 5:1 (mg/mg), 6:1 (mg/mg) to 5:1 (mg/mg), 10:1 (mg/mg) to 6:1 (mg/mg), 9:1 (mg/mg) to 6:1 (mg/mg), 8:1 (mg/mg) to 6:1 (mg/mg), 7:1 (mg/mg) to 6:1 (mg/mg), 10:1 (mg/mg) to 7:1 (mg/mg), 9:1 (mg/mg) to 7:1 (mg/mg), 8:1 (mg/mg) to 7:1 (mg/mg), 10:1 (mg/mg) to 8:1 (mg/mg), 9:1 (mg/mg) to 8:1 (mg/mg), or 10:1 (mg/mg) to 9:1 (mg/mg). In some embodiments, the ratio of HPMCAS to protein in the formulation is any of about 10:1 (mg/mg), 9:1 (mg/mg), 8:1 (mg/mg), 7:1 (mg/mg), 6:1 (mg/mg), 5:1 (mg/mg), 4:1 (mg/mg), 3:1 (mg/mg), 2:1 (mg/mg), 1:1 (mg/mg), 1:2 (mg/mg), 1:3 (mg/mg), 1:4 (mg/mg), 1:5 (mg/mg), 1:6 (mg/mg), 1:7 (mg/mg), 1:8 (mg/mg), 1:9 (mg/mg), or 1:10 (mg/mg).

In some embodiments, the formulation further comprises a histidine buffer (i.e., the formulation comprises a polypeptide, an HPMCAS, and a histidine buffer). In some embodiments, the histidine buffer is a histidine HCl buffer. In some embodiments, the histidine buffer (e.g., the histidine HCl buffer) is in the formulation at a concentration of about 1 mM to about 50 mM. In some embodiments, the histidine buffer in the formulation is at a concentration of between any of about 1 mM and 50 mM, 5 mM and 50 mM, 10 mM and 50 mM, 15 mM and 50 mM, 20 mM and 50 mM, 25 mM and 50 mM, 30 mM and 50 mM, 40 mM and 50 mM, 1 mM and 40 mM, 5 mM and 40 mM, 10 mM and 40 mM, 15 mM and 40 mM, 20 mM and 40 mM, 25 mM and 40 mM, 30 mM and 40 mM, 1 mM and 30 mM, 5 mM and 30 mM, 10 mM and 30 mM, 15 mM and 30 mM, 20 mM and 30 mM, 25 mM and 30 mM, 1 mM and 25 mM, 5 mM and 25 mM, 10 mM and 25 mM, 15 mM and 25 mM, 20 mM and 25 mM, 1 mM and 20 mM, 5 mM and 20 mM, 10 mM and 20 mM, 15 mM and 20 mM, 1 mM and 15 mM, 5 mM and 15 mM, 10 mM and 15 mM, 1 mM and 10 mM, 5 mM and 10 mM, 1 mM and 5 mM. In some embodiments, the histidine buffer is in the formulation at a concentration of any of about 1 mM, 5 mM, 10 mM, 15 mM, 20 mM, 25 mM, 30 mM, 40 mM, or 50 mM.

In some embodiments, the formulation further comprises a surfactant (i.e., the formulation comprises a polypeptide, an HPMCAS, a histidine buffer, and a surfactant). In some embodiments, the surfactant is a polysorbate. In some embodiments, the polysorbate is polysorbate 20. In some embodiments, the surfactant (e.g., polysorbate) in the formulation is at a concentration of about 0.005% (w/v) to about 1.0% (w/v). In some embodiments, the surfactant (e.g., a polysorbate such as polysorbate 20) in the formulation is at a concentration of between any of about 0.005% (w/v) to 1.0% (w/v), 0.01% (w/v) to 1.0% (w/v), 0.05% (w/v) to 1.0% (w/v), 0.1% (w/v) to 1.0% (w/v), 0.5% (w/v) to 1.0%, (w/v), 0.005% (w/v) to 0.5% (w/v), 0.01% (w/v) to 0.5% (w/v), 0.05% (w/v) to 0.5% (w/v), 0.1% (w/v) to 0.5% (w/v), 0.005% (w/v) to 0.1% (w/v), 0.01% (w/v) to 0.1% (w/v), 0.05% (w/v) to 0.1% (w/v), 0.005% (w/v) to 0.05% (w/v), 0.01% (w/v) to 0.05% (w/v), or 0.005% (w/v) to 0.01% (w/v). In some embodiments, the surfactant (e.g., a polysorbate such as polysorbate 20) in the formulation is at a concentration of any of about 0.005% (w/v), 0.01% (w/v), 0.05% (w/v), 0.1% (w/v), 0.5% (w/v), or 1% (w/v).

In some embodiments, the pH of the formulation is about 4.0 to about 9.0. In some embodiments, the pH of the formulation is between any of about 4.0 to 9.0, 5.0 to 9.0, 5.5 to 9.0, 6.0 to 9.0, 6.5 to 9.0, 7.0 to 9.0, 7.5 to 9.0, 8.0 to 9.0, 4.0 to 8.0, 5.0 to 8.0, 5.5 to 8.0, 6.0 to 8.0, 6.5 to 8.0, 7.0 to 8.0, 7.5 to 8.0, 4.0 to 7.5, 5.0 to 7.5, 5.5 to 7.5, 6.0 to 7.5, 6.5 to 7.5, 7.0 to 7.5, 4.0 to 7.0, 5.0 to 7.0, 5.5 to 7.0, 6.0 to 7.0, 6.5 to 7.0, 4.0 to 6.5 5.0 to 6.5, 5.5 to 6.5, 6.0 to 6.5, 4.0 to 6.0, 5.0 to 6.0, 5.5 to 6.0, 4.0 to 5.5, 5.0 to 5.5, or 4.0 to 5.0. In some embodiments, the pH of the formulation is any of about 4.0, 5.0, 5.3, 5.5, 6.0, 6.5, 7.0, 7.5, 8.0, or 9.0.

In some embodiments, the polypeptide of the formulation is an antibody. In some embodiments, the polypeptide of the formulation is a monoclonal antibody. In some embodiments, the polypeptide of the formulation is a human antibody, a chimeric antibody or a humanized antibody. In some embodiments, the polypeptide of the formulation is an antibody fragment selected from a Fab, Fab′-SH, Fv, scFv, and (Fab′)2 fragment. In some embodiments, the polypeptide is in the formulation at a concentration of about 5 mg/mL to about 100 mg/mL.

In some embodiments, the polypeptide (e.g., an antibody) is in the formulation at a concentration of between any of about 5 mg/mL to 100 mg/mL, 10 mg/mL to 100 mg/mL, 15 mg/mL to 100 mg/mL, 20 mg/mL to 100 mg/mL, 25 mg/mL to 100 mg/mL, 30 mg/mL to 100 mg/mL, 40 mg/mL to 100 mg/mL, 50 mg/mL to 100 mg/mL, 60 mg/mL to 100 mg/mL, 70 mg/mL to 100 mg/mL, 75 mg/mL to 100 mg/mL, 80 mg/mL to 100 mg/mL, 90 mg/mL to 100 mg/mL, 5 mg/mL to 90 mg/mL, 10 mg/mL to 90 mg/mL, 15 mg/mL to 90 mg/mL, 20 mg/mL to 90 mg/mL, 25 mg/mL to 90 mg/mL, 30 mg/mL to 90 mg/mL, 40 mg/mL to 90 mg/mL, 50 mg/mL to 90 mg/mL, 60 mg/mL to 90 mg/mL, 70 mg/mL to 90 mg/mL, 75 mg/mL to 90 mg/mL, 80 mg/mL to 90 mg/mL, 5 mg/mL to 80 mg/mL, 10 mg/mL to 80 mg/mL, 15 mg/mL to 80 mg/mL, 20 mg/mL to 90 mg/mL, 25 mg/mL to 80 mg/mL, 30 mg/mL to 80 mg/mL, 40 mg/mL to 80 mg/mL, 50 mg/mL to 80 mg/mL, 60 mg/mL to 80 mg/mL, 70 mg/mL to 80 mg/mL, 75 mg/mL to 80 mg/mL, 5 mg/mL to 75 mg/mL, 10 mg/mL to 75 mg/mL, 15 mg/mL to 75 mg/mL, 20 mg/mL to 75 mg/mL, 25 mg/mL to 75 mg/mL, 30 mg/mL to 75 mg/mL, 40 mg/mL to 75 mg/mL, 50 mg/mL to 75 mg/mL, 60 mg/mL to 75 mg/mL, 70 mg/mL to 75 mg/mL, 5 mg/mL to 70 mg/mL, 10 mg/mL to 70 mg/mL, 15 mg/mL to 70 mg/mL, 20 mg/mL to 70 mg/mL, 25 mg/mL to 70 mg/mL, 30 mg/mL to 70 mg/mL, 40 mg/mL to 70 mg/mL, 50 mg/mL to 70 mg/mL, 60 mg/mL to 70 mg/mL, 5 mg/mL to 65 mg/mL, 10 mg/mL to 65 mg/mL, 15 mg/mL to 65 mg/mL, 20 mg/mL to 65 mg/mL, 25 mg/mL to 65 mg/mL, 30 mg/mL to 65 mg/mL, 40 mg/mL to 65 mg/mL, 50 mg/mL to 65 mg/mL, 60 mg/mL to 65 mg/mL, 5 mg/mL to 60 mg/mL, 10 mg/mL to 60 mg/mL, 15 mg/mL to 60 mg/mL, 20 mg/mL to 60 mg/mL, 25 mg/mL to 60 mg/mL, 30 mg/mL to 60 mg/mL, 40 mg/mL to 60 mg/mL, 50 mg/mL to 60 mg/mL, 5 mg/mL to 50 mg/mL, 10 mg/mL to 50 mg/mL, 15 mg/mL to 50 mg/mL, 20 mg/mL to 50 mg/mL, 25 mg/mL to 50 mg/mL, 30 mg/mL to 50 mg/mL, 40 mg/mL to 50 mg/mL, 5 mg/mL to 40 mg/mL, 10 mg/mL to 40 mg/mL, 15 mg/mL to 40 mg/mL, 20 mg/mL to 40 mg/mL, 25 mg/mL to 40 mg/mL, 30 mg/mL to 40 mg/mL, 5 mg/mL to 30 mg/mL, 10 mg/mL to 30 mg/mL, 15 mg/mL to 30 mg/mL, 20 mg/mL to 30 mg/mL, 25 mg/mL to 30 mg/mL, 5 mg/mL to 25 mg/mL, 10 mg/mL to 25 mg/mL, 15 mg/mL to 25 mg/mL, 20 mg/mL to 25 mg/mL, 5 mg/mL to 20 mg/mL, 10 mg/mL to 20 mg/mL, 15 mg/mL to 20 mg/mL, 5 mg/mL to 15 mg/mL, 10 mg/mL to 15 mg/mL, or 5 mg/mL to 10 mg/mL. In some embodiments, the polypeptide (e.g., an antibody) is in the formulation at a concentration of any of about 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more than 100 mg/mL.

In some embodiments, the invention provides a formulation comprising a polypeptide (e.g., an antibody) at a concentration of about 5 mg/mL to about 50 mg/mL, an HPMCAS-LF, wherein the ratio of the HPMCAS-LF to polypeptide in the formulation is between about 1:1 (mg/mg) and 1:5 (mg/mg), a histidine-HCl buffer at a concentration of about 5 mM to about 20 mM, polysorbate 20 at a concentration of about 0.005% (w/v) to about 0.5% (w/v), and wherein the pH of the formulation is about 6.0 to about 7.0. In some embodiments, the invention provides a formulation comprising a polypeptide (e.g., an antibody) at a concentration of about 10 mg/mL, an HPMCAS-LF, wherein the ratio of the HPMCAS-LF to polypeptide in the formulation is about 1:1 (mg/mg), a histidine-HCl buffer at a concentration of about 10 mM, polysorbate 20 at a concentration of about 0.01% (w/v), and wherein the pH of the formulation is about 6.5.

In some embodiments, the invention provides a formulation comprising a polypeptide (e.g., an antibody) at a concentration of about 5 mg/mL to about 50 mg/mL, an HPMCAS-HF, wherein the ratio of the HPMCAS-HF to polypeptide in the formulation is between about 1:1 (mg/mg) and 1:5 (mg/mg), a histidine-HCl buffer at a concentration of about 5 mM to about 20 mM, polysorbate 20 at a concentration of about 0.005% (w/v) to about 0.5% (w/v), and wherein the pH of the formulation is about 6.0 to about 7.0. In some embodiments, the invention provides a formulation comprising a polypeptide (e.g., an antibody) at a concentration of about 10 mg/mL, an HPMCAS-HF, wherein the ratio of the HPMCAS-HF to polypeptide in the formulation is about 1:1 (mg:mg), a histidine-HCl buffer at a concentration of about 10 mM, polysorbate 20 at a concentration of about 0.01% (w/v), and wherein the pH of the formulation is about 6.5.

Spray Dried Formulations

In some aspects of the invention, the formulation of the invention is spray dried. In some embodiments, the invention provides a spray dried formulation comprising a polypeptide (e.g., an antibody) at a concentration of about 5 mg/mL to about 50 mg/mL, an HPMCAS-LF, wherein the ratio of the HPMCAS-LF to polypeptide in the formulation is between about 1:1 (mg/mg) and 1:5 (mg/mg), a histidine-HCl buffer at a concentration of about 5 mM to about 20 mM, polysorbate 20 at a concentration of about 0.005% (w/v) to about 0.5% (w/v), and wherein the pH of the formulation is about 6.0 to about 7.0. In some embodiments, the invention provides a spray dried formulation comprising a polypeptide (e.g., an antibody) at a concentration of about 10 mg/mL, an HPMCAS-LF, wherein the ratio of the HPMCAS-LF to polypeptide in the formulation is about 1:1 (mg/mg), a histidine-HCl buffer at a concentration of about 10 mM, polysorbate 20 at a concentration of about 0.01% (w/v), and wherein the pH of the formulation is about 6.5.

In some embodiments, the invention provides a spray dried formulation comprising a polypeptide (e.g., an antibody) at a concentration of about 5 mg/mL to about 50 mg/mL, an HPMCAS-HF, wherein the ratio of the HPMCAS-HF to polypeptide in the formulation is between about 1:1 (mg/mg) and 1:5 (mg/mg), a histidine-HCl buffer at a concentration of about 5 mM to about 20 mM, polysorbate 20 at a concentration of about 0.005% (w/v) to about 0.5% (w/v), and wherein the pH of the formulation is about 6.0 to about 7.0. In some embodiments, the invention provides a spray dried formulation comprising a polypeptide (e.g., an antibody) at a concentration of about 10 mg/mL, an HPMCAS-HF, wherein the ratio of the HPMCAS-HF to polypeptide in the formulation is about 1:1 (mg/mg), a histidine-HCl buffer at a concentration of about 10 mM, polysorbate 20 at a concentration of about 0.01% (w/v), and wherein the pH of the formulation is about 6.5.

In some embodiments, the polypeptide (e.g., an antibody) of the spray dried formulation is stable at about 90° C. for at least about 15 minutes to at least about 10 hours. In some embodiments, the polypeptide of the spray dried formulation is stable at about 90° C. for between any of about 15 minutes and 10 hours, 30 minutes and 10 hours, 45 minutes and 10 hours, 1 hour and 10 hours, 2 hours and 10 hours, 3 hours and 10 hours, 4 hours and 10 hours, 5 hours and 10 hours, 6 hours and 10 hours, 7 hours and 10 hours, 8 hours and 10 hours, 9 hours and 10 hours. In some embodiments, the polypeptide of the spray dried formulation is stable at about 90° C. for any of at least about 15 minutes, 30 minutes, 45 minutes, 1 hour, 2 hours, 3 hours, 4 hours, 5 hours, 6 hours, 7 hours, 8 hours, 9 hours, or 10 hours.

In some embodiments, the polypeptide (e.g., an antibody) of the spray dried formulation is stable at about 37° C. for at least about 1 week to more than 6 weeks. In some embodiments, the polypeptide of the spray dried formulation is stable at about 37° C. for any of between about 1 week and 6 weeks, 2 weeks and 6 weeks, 3 weeks and 6 weeks, 4 weeks and 6 weeks, or 5 weeks and 6 weeks. In some embodiments, the polypeptide of the spray dried formulation is stable at about 37° C. for any of about 1 week, 2 weeks, 3 weeks, 4 weeks, 5 weeks, 6 weeks, or more than 6 weeks.

In some embodiments of the invention, the polypeptide (e.g., an antibody) in the formulation shows reduced aggregation and/or reduced chemical degradation. In some embodiments of the invention, the polypeptide in the formulation shows reduced aggregation and/or reduced chemical degradation compared to a similar formulation that does not include an HPMCAS. In some embodiments, the reduced chemical degradation comprises reduced formation of succinimide variants of the polypeptide and/or reduced formation of pyroglutamate variants of the polypeptide.

In some embodiments, the spray dried formulation is a powder. In some embodiments, the spray dried formulation provides an amorphous formulation of the polypeptide. In some embodiments, the spray dried formulation provides an amorphous glassy formulation of the polypeptide. An amorphous formulation is a non-crystalline formulation that possesses no long-range order. In some embodiments, a glass is a rigid inert matrix that can be measured, for example, by glass transition temperature.

Encapsulated Forms of Polypeptides

In some embodiments, the invention provides encapsulated forms of the polypeptide. In some embodiments, the spray dried formulation of the invention is encapsulated in an acid polymer system. In some embodiments, the acid polymer system is a lactic acid/glycolic acid polymer system. In some embodiments the acid polymer system is a poly(lactic-co-glycolic acid) (PLGA) system. In some embodiments, the acid polymer system generates a low pH microenvironment for the spray dried polypeptide. In some embodiments, the low pH microenvironments is about pH 3 to about pH 5. In some embodiments, the low pH microenvironment is less than about pH 5. In some embodiments, the low pH microenvironment is less than about pH 6. In some embodiments, the low pH microenvironment is less than about pH 7. Without being bound by theory, the presence of the HPMCAS in the spray dried formulation stabilizes the polypeptide in the low pH microenvironment created when encapsulating the spray dried polypeptide.

In some embodiments, the spray dried formulation of the polypeptide is encapsulated in PLGA. In some embodiments, the spray dried formulation of the polypeptide is encapsulated in a PLGA rod.

Methods of Producing Formulations of Polypeptides

In some embodiments, the invention provided methods of producing formulations comprising adding a hydroxypropyl methylcellulose acetate succinate (HPMCAS) to a polypeptide formulation. In some embodiments, the HPMCAS comprises about 8% acetate and about 15% succinate (HPMCAS-LF) or about 12% acetate and about 7% succinate (HPMCAS-HF). In some embodiments, the ratio of the HPMCAS to protein in the formulation is about 1:1 (mg/mg) to about 1:5 (mg/mg). In some embodiments, a histidine buffer (e.g., a histidine HCl buffer) is added to the formulation. In some embodiments, the histidine buffer is added to the formulation at a concentration of about 5 mM to about 20 mM. In some embodiments, the histidine buffer is added to the formulation at a concentration of about 10 mM. In some embodiments, a polysorbate (e.g., polysorbate 20) is added to the formulation. In some embodiments, the polysorbate is added to the formulation at a concentration of about 0.005% (w/v) to about 0.5% (w/v). In some embodiments, the polysorbate is added to the formulation at a concentration of about 0.01% (w/v). In some embodiments, the pH of the formulation is adjusted to about 6.0 to about 7.0. In some embodiments, the pH of the formulation is adjusted to about 6.5. In some embodiments, the polypeptide (e.g., an antibody) is added to the formulation at a concentration of about 5 mg/mL to about 100 mg/mL or about 10 mg/mL to about 80 mg/mL. In some embodiments, the polypeptide (e.g., an antibody) is added to the formulation at a concentration of about 10 mg/mL.

Additional Aspects of Formulations

In some embodiments, the polypeptide in the polypeptide formulation maintains functional activity.

The formulations to be used for in vivo administration must be sterile. This is readily accomplished by filtration through sterile filtration membranes.

The formulations herein may also contain more than one active compound as necessary for the particular indication being treated, preferably those with complementary activities that do not adversely affect each other. For example, in addition to a polypeptide, it may be desirable to include in the one formulation, an additional polypeptide (e.g., antibody). Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

### Spray Drying

In some aspects, the invention provides spray dried formulation of a polypeptide. In some embodiments, the formulations comprising a polypeptide and HPMCAS as described herein is spray dried. Spray drying is a method of producing a dry powder from a liquid or slurry by rapidly drying with a hot gas. In some embodiments, spray drying is a method of drying of thermally-sensitive materials such as pharmaceuticals (e.g., a polypeptide such as an antibody) and/or materials which may require extremely consistent, fine, particle size. Spray drying as used herein is distinct from freeze drying commonly used to prepare monoclonal antibody formulations as it is performed at temperatures above ambient temperature. Spray drying temperatures are commonly expressed as “air inlet” and “air outlet” temperatures. In one embodiment, the spray drying is performed at an air inlet temperature from about 100° C. to about 220° C. (for example from about 120° C. to about 160° C.) and an air outlet temperature from about 50° C. to about 100° C. (for example from about 60° C. to about 80° C., about 100° C. or about 120° C.). In some embodiments, the air inlet temperature is about 110° C. and the air outlet temperature is about 60° C. In some embodiments, the air inlet temperature is about 80° C. to about 220° C. and the air outlet temperature is about 50° C. to about 100° C. In some embodiments, the air inlet temperature is between any of about 80° C. and about 220° C., about 80° C. and about 180° C., about 80° C. and about 160° C., about 80° C. and about 140° C., about 90° C. and about 220° C., about 90° C. and about 180° C., about 90° C. and about 160° C., about 90° C. and about 140° C., about 100° C. and about 220° C., about 100° C. and about 180° C., about 100° C. and about 160° C., about 100° C. and about 140° C., about 110° C. and about 220° C., about 110° C. and about 180° C., about 110° C. and about 160° C., or about 110° C. and about 140° C. In some embodiments, the air inlet temperature is any of about 80° C., 90° C., 100° C., 110° C., 120° C., 130° C., 140° C., 150° C., 160° C., 170° C., 180° C., 190° C., 200° C., 210° C., or 220° C. In some embodiments, the air outlet temperatures is between any of about 50° C. to about 100° C., 50° C. to about 90° C., 50° C. to about 80° C., 50° C. to about 70° C., 50° C. to about 60° C., about 60° C. to about 100° C., 60° C. to about 90° C., 60° C. to about 80° C., 60° C. to about 70° C., about 70° C. to about 100° C., 70° C. to about 90° C., 70° C. to about 80° C., about 80° C. to about 100° C., 80° C. to about 90° C., or about 90° C. to about 100° C. In some embodiments, the air outlet temperature is about 50° C., about 60° C., about 70° C., about 80° C., about 90° C., or about 100° C.

The spray drying process generally comprises: atomization of the liquid feed; drying of the droplets; and separation or recovery of the dried product.

In some embodiments, the atomizers used in the invention herein include but are not limited to rotary atomizers, pneumatic nozzle atomizers, ultrasonic nozzle atomizers, and sonic nozzles.

The contact between the liquid feed and the drying air can occur in two different modes. In a co-current system, drying air and particles (droplets) move through the drying chamber in the same direction. When drying air and droplets move in an opposite direction, this is called a counter-current mode. Particles produced in counter-current mode usually show a higher temperature than the exhausting air. The exhausted air itself can leave the system or can be recirculated. By choosing from the various spray dryer designs (size, atomizer, aseptic conditions, etc.) and adjusting the different process parameters (drying air flow, drying air temperature, etc.), the final powder properties like particle size, shape and structure or even sterility can be modified. If the resulting moisture of the recovered powder is not sufficiently low, post-treatment might be required, e.g., in the form of secondary drying (e.g., using a benchtop lyophilizer), fluid bed dryers and coolers, contact dryers or even microwave dryers.

When the liquid feed is atomized, its surface area to mass ratio is increased, the heat transfer between the air and the droplets is accelerated, and droplets can dry relatively rapidly. Two convection processes may be involved: heat transfer (air to droplet) and mass transfer of moisture (droplet to air). In the latter, moisture permeates through the boundary layer that surrounds each droplet. Transfer rates may be influenced by temperature, humidity, transport properties of the surrounding air, droplet diameter and relative velocity between droplet and air.

The last step of a spray drying process is typically the separation of the powder from the air/gas and the removal of the dried product. In some embodiments, this step is as effective as possible to obtain high powder yields and to prevent air pollution through powder emission to the atmosphere. To this end, various methods are available such as cyclones, bag filters, electrostatic precipitators, high pressure gas, electrostatic charge and combinations thereof.

The spray drying process produces particles comprising the polypeptide (e.g., an antibody).

In one embodiment, the characteristics of the spray dried powder comprise any one or more or the following:
(a) average particle size: from about 2 microns to about 30 microns; e.g. from about 2 microns to about 10 microns;
(b) particle morphology: predominantly spherical particles, collapsed spheres, some dimples or holes in particles, “dry raisin” shape;
(c) water content: less than about 10%, for example less than about 5%, e.g., where water content is measured by a chemical titration method (e.g. Karl Fischer method) or a weight-loss method (high-temperature heating); and
(d) stability: e.g., assessed by suspending the particles in a vehicle and evaluating physical stability and/or chemical stability and/or biological activity of the suspension preparation. In one embodiment, the percent monomer of such preparation is 95% to 100%, e.g. as evaluated by size exclusion chromatography (SEC).

### Controlled Release Formulations of Polypeptides

A slow-release formulation in accordance with the invention typically comprises a polypeptide (e.g., an antibody) formulated with HPMCAS as described herein, a polymeric carrier, and a release modifier for modifying a release rate of the polypeptide from the polymeric carrier. By varying the manufacturing conditions of polymer-based delivery compositions, the release kinetic properties of the resulting compositions can be modulated. The polymeric carrier usually comprises one or more biodegradable polymers or co-polymers or combinations thereof. For example, the polymeric carrier may be selected from poly-lactic acid (PLA), poly-glycolic acid (PGA), polylactide-co-glycolide (PLGA), polyesters, poly(orthoester), poly(phosphazine), poly(phosphate ester), polyethylene glycol (PEG), polycaprolactones, or a combination thereof. Other polymers for use in the formulations of the invention include but are not limited to poly(meth)acrylic acid derivatives, insoluble cellulose derivatives, poly vinyl acetate, hydroxypropyl cellulose (HPC), polyethylene oxide (PEO), polyvinyl alcohol (PVA), polyvinylpyrrolidone (PVP), poloxamer, sodium carboxymethyl cellulose (NaCMC), poly(acrylic acid), polyvinyl acetyl phthalate (PVAP), polysaccharides (e.g., sodium alginate, carrageenan, guar and xanthan gum), poly(meth) acrylates, Eudragit® L, Eudragit® L, 100-55, Eudragit® L 30D-55, Eudragit® L 100, Eudragit® S, Eudragit® S100, Eudragit® FS, Eudragit® FS 30D, Carbopol®, Carbopol® 934P, Carbopol® 940, Carbopol® 974P, Carbopol® 971 (Lubrizol), and Noveon® AA-1. In some embodiments, the formulation comprises a polymer described in Maskova, E. et al., J. Controlled Release, 2020, 324:695-727, incorporated herein in its entirety.

In certain embodiments, the polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oleyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

In certain embodiments, the polypeptide (e.g., an antibody) formulated with HPMCAS is incorporated into a microsphere-based sustained release composition. In certain embodiments, the microspheres are prepared from PLGA. The amount of the polypeptide incorporated in the microspheres and the release rate of the polypeptide can be controlled by varying the conditions used for preparing the microspheres. Processes for producing such slow-release formulations are described in US 2005/0281861 and US 2008/0107694.

In certain embodiments, the polypeptide formulated with HPMCAS is incorporated into a biodegradable implant (such as a microneedle). Matrix implants (such as microneedles) are typically used to treat ocular diseases that require a loading dose followed by tapering doses of the drug during a 1-day to 6-month time period (Davis et al. (2004) Curr Opin Mol Therap 6, 195-205). They are most commonly made from the copolymers poly-lactic-acid (PLA) and/or poly-lactic-glycolic acid (PLGA), which degrade to water and carbon dioxide. The rate and extent of drug release from the implant can be decreased by altering the relative concentrations of lactide (slow) and glycolide (fast), altering the polymer weight ratios, adding additional coats of polymer, or using hydrophobic, insoluble drugs. The release of drug generally follows first-order kinetics with an initial burst of drug release followed by a rapid decline in drug levels. Biodegradable implants do not require removal, as they dissolve over time (Hsu (2007) Curr Opin Ophthalmol 18, 235-9). Biodegradable implants also allow flexibility in dose and treatment from short duration (weeks) to longer duration (months to a year), depending on the polymer PLA/PLGA ratio, which is another benefit in tailoring drug delivery to disease progression, because dose and treatment requirements may change over time.

The sustained-release formulations of polypeptides were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis. “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

In some embodiments, the controlled release formulation of the polypeptide is produced by a hot melt extrusion process. For example, hot melt extrusion can be performed using a HAAKE™ MiniCTW Micro-Conical Twin Screw Compounder (Thermo Scientific, Karlsruhe, Germany) with a barrel capacity of 5 g or 7 cm³. In some embodiments, the extruder barrel is pre-heated to 90° C. and screw speed is set to 40 rpm. In some embodiments, the front barrel opening of the extruder is kept closed in the recirculation mode. In some embodiments, a pre-weighed solid polymer and spray dried polypeptide are manually fed slowly into the extruder and re-circulated back to the barrel for micro-compounding. In some embodiments, the polypeptide loading for implants uses a spray dried formulation fixed about 1% to about 30%, about 1% to about 20%, about 1% to about 15%, about 5% to about 30%, about 5% to about 20%, about 5% to about 15%, about 10% to about 30%, about 10% to about 20%, about 10% to about 15% (w/w) polypeptide/PLGA. In some embodiments, the polypeptide loading for implants uses a spray dried formulation fixed at any of about 1%, 5%, 10%, 15%, 20% or 30% (w/w) polypeptide/PLGA. In some embodiments, the polypeptide loading for implants uses a spray dried formulation fixed at about 15% (w/w) polypeptide/PLGA. In some embodiments, the melt-phase blending is continued for about 30 min under re-circulation mode after complete feeding and then extruded through an about 0.5 mm circular die. In some embodiments, the extrudates are cooled to room temperature, cut to cylinders of desired length and stored at 2-8° C. until further use.

### Hydroxypropyl Methylcellulose Acetate Succinate (HPMCAS)

In some aspects, the invention provides formulations of a polypeptide and an HPMCAS. In some embodiments, the HPMCAS provides protection to the polypeptide during high temperature spray drying processes as well as against the low pH microenvironments during acid polymer encapsulation.

The reaction of HMPC with acetic acid and succinic acid produced the HPMC derivatives known as hydroxypropyl methylcellulose acetate succinate (referred to as HPMCAS) in which the hydroxyl groups in HPMC were modified to form the respective esters. Reaction with acetic acid converts the hydroxyl group in HPMC into acetyl group whereas the reaction of succinic acid converts the hydroxyl group in HPMC into succinyl group. In some embodiments, the HMPC is an HPMC derivative. In some embodiments, the HPMC comprises 8% acetic acid (referred to as HPMCAS-LF). In some embodiments, the HPMC comprises 7% succinic acid (referred to as HPMCAS-HF). The acetyl and succinoyl substitution levels have a significant impact on the solubility enhancement effect of HPMCAS when used as a drug carrier. Due to an acid dissociation constant (pKa) value of about 5 of succinate groups of HPMCAS, the polymer is predominantly un-ionized at pH values below 4 and is partially to completely ionized at pH values of about 5 or higher. Thus, HPMC derivatives are polymeric buffers with succinic acid. In its un-ionized state (pH<5), HPMCAS is insoluble in water and around neutral (pH 6.0-7.5) conditions it forms stable colloidal assemblies that are water soluble. It is a solubility enhancer for poorly soluble drugs (Friesen, D T et al., Mol Pharm, 2008. 5(6):1003-19), is insoluble in gastric fluid and rapidly dissolves in the small intestine (Curatolo, W et al., Pharm Res, 2009, 26(6):1419-31). Although HPMCAS based dispersions have been suitable for small molecule based sustained drug release applications, their usage with large protein therapeutics have not been explored previously. HPMCAS in spray dried powder form is insoluble below pH 6, starts to be soluble around pH 6 to pH 6.5 and completely soluble at pH>=6.5. Thus, the advantage is that HPMCAS would provide protection by coating the protein in the spray dried form (thus still remaining as SD powder) and prevent it from interacting with the acidic counter ions in a low pH microenvironment. Consequently, we have tried to take advantage of the low pH insolubility of HPMCAS for use as a spray dried drug carrier inside the acidic microclimate of a PLGA system. However, almost all of today's delivery systems using HPMCAS are administered orally for small molecule drugs, and there has been no research showing their possible use in delivery for large proteins (Woodley, J F, Crit Rev Ther Drug Carrier Syst, 1994, 11(2-3):61-95). Specifically, HPMCAS formulations described here are a new strategy for stabilizing protein, without obvious implications for any specific route of delivery. Moreover, HPMCAS in its un-ionized state with a high glass transition (>100° C.) could fit as an excipient for use in spray dried protein therapeutics as well as for high temperature manufacturing of drug delivery systems.

A review of HPMC an excipient for oral and oromucosal drug delivery is provided by Maskova, E. et al., J. Controlled Release, 2020, 324:695-727.

### Polypeptides

Examples of polypeptides used in the formulations described herein include but are not limited to immunoglobulins, immunoadhesins, antibodies, enzymes, hormones, fusion proteins, Fc-containing proteins, immunoconjugates, cytokines and interleukins. Examples of polypeptide include, but are not limited to, mammalian proteins, such as, e.g., renin; a hormone; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; an enzyme; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-b; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); a cytokine; CD proteins such as CD3, CD4, CD8, CD19 and CD20; erythropoietin; osteoinductive factors; immunotoxins; a fusion polypeptide, i.e. a polypeptide comprised on two or more heterologous polypeptides or fragments thereof and encoded by a recombinant nucleic acid; an Fc-containing polypeptide, for example, a fusion protein comprising an immunoglobulin Fc region, or fragment thereof, fused to a second polypeptide; an immunoconjugate; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as CA125 (ovarian cancer antigen) or HER2, HER3 or HER4 receptor; immunoadhesins; and fragments and/or variants of any of the above-listed proteins as well as antibodies, including antibody fragments, binding to a protein, including, for example, any of the above-listed proteins.

### Antibodies

In some embodiments of any of the methods described herein, the polypeptide or the formulations described herein is an antibody.

Molecular targets for antibodies include CD proteins and their ligands, such as, but not limited to: (i) CD3, CD4, CD8, CD19, CD11a, CD20, CD22, CD34, CD40, CD79a (CD79a), and CD79β (CD79b); (ii) members of the ErbB receptor family such as the EGF receptor, HER2, HER3 or HER4 receptor; (iii) cell adhesion molecules such as LFA-1, Mac1, p150,95, VLA-4, ICAM-1, VCAM and αv/β3 integrin, including either alpha or beta subunits thereof (e.g., anti-CD11a, anti-CD18 or anti-CD11b antibodies); (iv) growth factors such as VEGF; IgE; blood group antigens; flk2/flt3 receptor; obesity (OB) receptor; mpl receptor; CTLA-4; protein C, BR3, c-met, tissue factor, β7, etc.; and (v) cell surface and transmembrane tumor-associated antigens (TAA), such as those described in U.S. Pat. No. 7,521,541.

Other exemplary antibodies include those selected from, and without limitation, anti-CD20 antibody, anti-CD40 antibody, anti-HER2 antibody, anti-IL6 antibody, anti-IgE antibody, anti-IL13 antibody, anti-Flu A antibody, anti-TIGIT antibody, anti-PD-L1 antibody, anti-VEGF-A antibody, anti-VEGF-A/ANG2 antibody, anti-CD79b antibody, anti-ST2 antibody, anti-factor D antibody, anti-factor IX antibody, anti-factor X antibody, anti-abeta antibody, anti-tau antibody, anti-CEA antibody, anti-CEA/CD3 antibody, anti-CD20/CD3 antibody, anti-FcRH5/CD3 antibody, anti-Her2/CD3 antibody, anti-FGFR1/KLB antibody, a FAP-4-1 BBL fusion protein, a FAP-IL2v fusion protein, anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD10 antibody, anti-CD11a antibody, anti-CD11c antibody, anti-CD13 antibody, anti-CD14 antibody, anti-CD15 antibody, anti-CD19 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCA/CD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokeratins antibody, anti-vimentins antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody and anti-Tn-antigen antibody.

Polyclonal Antibodies

In some embodiments, the antibodies are polyclonal antibodies. Polyclonal antibodies are preferably raised in animals by multiple subcutaneous (sc) or intraperitoneal (ip) injections of the relevant antigen and an adjuvant. It may be useful to conjugate the relevant antigen to a polypeptide that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the polypeptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. In some embodiments, the animal is boosted with the conjugate of the same antigen, but conjugated to a different polypeptide and/or through a different cross-linking reagent. Conjugates also can be made in recombinant cell culture as polypeptide fusions. Also, aggregating agents such as alum are suitably used to enhance the immune response.

Monoclonal Antibodies

In some embodiments, the antibodies purified on reusable chromatography material cleaned by the methods of the invention are monoclonal antibodies. Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature 256:495 (1975), or may be made by recombinant DNA methods (U.S. Pat. No. 4,816,567).

In the hybridoma method, a mouse or other appropriate host animal, such as a hamster, is immunized as herein described to elicit lymphocytes that produce or are capable of producing antibodies that will specifically bind to the polypeptide used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from MOPC-21 and MPC-11 mouse tumors available from the Salk Institute Cell Distribution Center, San Diego, California USA, and SP-2 or X63-Ag8-653 cells available from the American Type Culture Collection, Rockville, Maryland USA. Human myeloma and mouse-human heteromyeloma cell lines also have been described for the production of human monoclonal antibodies (Kozbor, J. Immunol. 133:3001 (1984); Brodeur et al., Monoclonal Antibody Production Techniques and Applications pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem. 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, polypeptide A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin polypeptide, to obtain the synthesis of monoclonal antibodies in the recombinant host cells. Review articles on recombinant expression in bacteria of DNA encoding the antibody include Skerra et al., Curr. Opinion in Immunol. 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature 348:552-554 (1990). Clackson et al., Nature 352:624-628 (1991) and Marks et al., J. Mol. Biol. 222:581-597 (1991) describe the isolation of murine and human antibodies, respectively, using phage libraries. Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison et al., Proc. Natl Acad. Sci. USA 81:6851 (1984)), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In some embodiments of any of the methods described herein, the antibody is IgA, IgD, IgE, IgG, or IgM. In some embodiments, the antibody is an IgG monoclonal antibody.

Humanized Antibodies

In some embodiments, the antibody is a humanized antibody. Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol. 151:2296 (1993); Chothia et al., J. Mol. Biol. 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA 89:4285 (1992); Presta et al., J. Immunol. 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

In some embodiments, the antibody is a human antibody. As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immuno. 7:33 (1993); and U.S. Pat. Nos. 5,591,669; 5,589,369; and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat polypeptide gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

Human antibodies may also be generated by in vitro activated B cells (see U.S. Pat. Nos. 5,567,610 and 5,229,275).

Antibody Fragments

In some embodiments, the antibody is an antibody fragment. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody,” e.g., as described in U.S. Pat. No. 5,641,870 for example. Such linear antibody fragments may be monospecific or bispecific.

In some embodiments, fragments of the antibodies described herein are provided. In some embodiments, the antibody fragment is an antigen binding fragment. In some embodiments, the antigen binding fragment is selected from the group consisting of a Fab fragment, a Fab′ fragment, a F(ab′)₂ fragment, a scFv, a di-scFv, a bi-scFv, a tandem (di, tri)-scFv, a Fv, a sdAb, a tri-functional antibody, a BiTE, a diabody and a triabody.

Bispecific Antibodies

In some embodiments, the antibody is a bispecific antibody. Bispecific antibodies are antibodies that have binding specificities for at least two different epitopes. Exemplary bispecific antibodies may bind to two different epitopes. Alternatively, a bispecific antibody binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the cell. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. In some embodiments, the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers that are recovered from recombinant cell culture. In some embodiments, the interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152:5368 (1994).

Antibodies with more than two valencies are contemplated. For example, trispecific antibodies can be prepared. Tutt et al., J. Immunol. 147: 60 (1991).

Multivalent Antibodies

In some embodiments, the antibodies are multivalent antibodies. A multivalent antibody may be internalized (and/or catabolized) faster than a bivalent antibody by a cell expressing an antigen to which the antibodies bind. The antibodies provided herein can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)n-VD2-(X2) n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: V_{H}-CH1-flexible linker-V_{H}-CH1-Fc region chain; or V_{H}-CH1-V_{H}-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In some embodiments, the antibody is a multispecific antibody. Example of multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. In some embodiment that antibody has polyepitopic specificity; for example, the ability to specifically bind to two or more different epitopes on the same or different target(s). In some embodiments, the antibodies are monospecific; for example, an antibody that binds only one epitope. According to one embodiment the multispecific antibody is an IgG antibody that binds to each epitope with an affinity of 5 ┌M to 0.001 pM, 3 ┌M to 0.001 pM, 1 ┌M to 0.001 pM, 0.5 ┌M to 0.001 pM, or 0.1 ┌M to 0.001 pM.

Other Antibody Modifications

It may be desirable to modify the antibody provided herein with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J., Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement mediated lysis and ADCC capabilities. See Stevenson et al., Anti-Cancer Drug Design 3:219-230 (1989).

For increasing the serum half-life of the antibody, amino acid alterations can be made in the antibody as described in US 2006/0067930, which is hereby incorporated by reference in its entirety.

Polypeptide Variants and Modifications

Amino acid sequence modification(s) of the polypeptides, including antibodies, described herein may be used in reusable chromatography material cleaned by the methods of described herein.

Variant Polypeptides

“Polypeptide variant” means a polypeptide, preferably an active polypeptide, as defined herein having at least about 80% amino acid sequence identity with a full-length native sequence of the polypeptide, a polypeptide sequence lacking the signal peptide, an extracellular domain of a polypeptide, with or without the signal peptide. Such polypeptide variants include, for instance, polypeptides wherein one or more amino acid residues are added, or deleted, at the N or C-terminus of the full-length native amino acid sequence. Ordinarily, a TAT polypeptide variant will have at least about 80% amino acid sequence identity, alternatively at least about any of 85%, 90%, 95%, 96%, 97%, 98%, or 99% amino acid sequence identity, to a full-length native sequence polypeptide sequence, a polypeptide sequence lacking the signal peptide, an extracellular domain of a polypeptide, with or without the signal peptide. Optionally, variant polypeptides will have no more than one conservative amino acid substitution as compared to the native polypeptide sequence, alternatively no more than about any of 2, 3, 4, 5, 6, 7, 8, 9, or 10 conservative amino acid substitutions as compared to the native polypeptide sequence.

The variant polypeptide may be truncated at the N-terminus or C-terminus, or may lack internal residues, for example, when compared with a full length native polypeptide. Certain variant polypeptides may lack amino acid residues that are not essential for a desired biological activity. These variant polypeptides with truncations, deletions, and insertions may be prepared by any of a number of conventional techniques. Desired variant polypeptides may be chemically synthesized. Another suitable technique involves isolating and amplifying a nucleic acid fragment encoding a desired variant polypeptide, by polymerase chain reaction (PCR). Oligonucleotides that define the desired termini of the nucleic acid fragment are employed at the 5′ and 3′ primers in the PCR. Preferably, variant polypeptides share at least one biological and/or immunological activity with the native polypeptide disclosed herein.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

For example, it may be desirable to improve the binding affinity and/or other biological properties of the polypeptide. Amino acid sequence variants of the polypeptide are prepared by introducing appropriate nucleotide changes into the antibody nucleic acid, or by peptide synthesis. Such modifications include, for example, deletions from, and/or insertions into and/or substitutions of, residues within the amino acid sequences of the polypeptide. Any combination of deletion, insertion, and substitution is made to arrive at the final construct, provided that the final construct possesses the desired characteristics. The amino acid changes also may alter post-translational processes of the polypeptide (e.g., antibody), such as changing the number or position of glycosylation sites.

Guidance in determining which amino acid residue may be inserted, substituted or deleted without adversely affecting the desired activity may be found by comparing the sequence of the polypeptide with that of homologous known polypeptide molecules and minimizing the number of amino acid sequence changes made in regions of high homology.

A useful method for identification of certain residues or regions of the polypeptide (e.g., antibody) that are preferred locations for mutagenesis is called “alanine scanning mutagenesis” as described by Cunningham and Wells, Science 244:1081-1085 (1989). Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table 1 below under the heading of “preferred substitutions.” If such substitutions result in a change in biological activity, then more substantial changes, denominated “exemplary substitutions” in the Table A, or as further described below in reference to amino acid classes, may be introduced and the products screened.

TABLE A
Exemplary Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, Biochemistry second ed. Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the polypeptide to improve its stability (particularly where the antibody is an antibody fragment such as an Fv fragment).

A particularly preferred type of substitutional variant involves substituting one or more hypervariable region residues of a parent antibody (e.g., a humanized antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and target. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the polypeptide alters the original glycosylation pattern of the antibody. The polypeptide may comprise non-amino acid moieties. For example, the polypeptide may be glycosylated. Such glycosylation may occur naturally during expression of the polypeptide in the host cell or host organism, or may be a deliberate modification arising from human intervention. By altering is meant deleting one or more carbohydrate moieties found in the polypeptide, and/or adding one or more glycosylation sites that are not present in the polypeptide.

Glycosylation of polypeptide is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Removal of carbohydrate moieties present on the polypeptide may be accomplished chemically or enzymatically or by mutational substitution of codons encoding for amino acid residues that serve as targets for glycosylation. Enzymatic cleavage of carbohydrate moieties on polypeptides can be achieved by the use of a variety of endo- and exo-glycosidases.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains, acetylation of the N-terminal amine, and amidation of any C-terminal carboxyl group.

Chimeric Polypeptides

The polypeptide described herein may be modified in a way to form chimeric molecules comprising the polypeptide fused to another, heterologous polypeptide or amino acid sequence. In some embodiments, a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag.

In an alternative embodiment, the chimeric molecule may comprise a fusion of the polypeptide with an immunoglobulin or a particular region of an immunoglobulin. A bivalent form of the chimeric molecule is referred to as an “immunoadhesin.”

As used herein, the term “immunoadhesin” designates antibody-like molecules which combine the binding specificity of a heterologous polypeptide with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The Ig fusions preferably include the substitution of a soluble (transmembrane domain deleted or inactivated) form of a polypeptide in place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule.

Polypeptide Conjugates

The polypeptide for use in polypeptide formulations may be conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such conjugates can be used. In addition, enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated polypeptides. Examples include ²¹²Bi, ¹³¹I, ¹³¹In, ⁹⁰Y, and ¹⁸⁶Re. Conjugates of the polypeptide and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the polypeptide.

Conjugates of a polypeptide and one or more small molecule toxins, such as a calicheamicin, maytansinoids, a trichothene, and CC1065, and the derivatives of these toxins that have toxin activity, are also contemplated herein. Maytansinoids are mitototic inhibitors which act by inhibiting tubulin polymerization. Maytansine was first isolated from the east African shrub Maytenus serrata. Subsequently, it was discovered that certain microbes also produce maytansinoids, such as maytansinol and C-3 maytansinol esters. Synthetic maytansinol and derivatives and analogues thereof are also contemplated. There are many linking groups known in the art for making polypeptide-maytansinoid conjugates, including, for example, those disclosed in U.S. Pat. No. 5,208,020. The linking groups include disufide groups, thioether groups, acid labile groups, photolabile groups, peptidase labile groups, or esterase labile groups, as disclosed in the above-identified patents, disulfide and thioether groups being preferred.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Another conjugate of interest comprises a polypeptide conjugated to one or more calicheamicin molecules. The calicheamicin family of antibiotics are capable of producing double-stranded DNA breaks at sub-picomolar concentrations. For the preparation of conjugates of the calicheamicin family, see, e.g., U.S. Pat. No. 5,712,374. Structural analogues of calicheamicin which may be used include, but are not limited to, γ₁^{I}, α₂^{I}, α₃^{I}, N-acetyl-γ₁^{I}, PSAG and θ₁^{I}. Another anti-tumor drug that the antibody can be conjugated is QFA which is an antifolate. Both calicheamicin and QFA have intracellular sites of action and do not readily cross the plasma membrane. Therefore, cellular uptake of these agents through polypeptide (e.g., antibody) mediated internalization greatly enhances their cytotoxic effects.

Other antitumor agents that can be conjugated to the polypeptides described herein include BCNU, streptozoicin, vincristine and 5-fluorouracil, the family of agents known collectively LL-E33288 complex, as well as esperamicins.

In some embodiments, the polypeptide may be a conjugate between a polypeptide and a compound with nucleolytic activity (e.g., a ribonuclease or a DNA endonuclease such as a deoxyribonuclease; DNase).

In yet another embodiment, the polypeptide (e.g., antibody) may be conjugated to a “receptor” (such streptavidin) for utilization in tumor pre-targeting wherein the polypeptide receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

In some embodiments, the polypeptide may be conjugated to a prodrug-activating enzyme which converts a prodrug (e.g., a peptidyl chemotherapeutic agent) to an active anti-cancer drug. The enzyme component of the immunoconjugate includes any enzyme capable of acting on a prodrug in such a way so as to convert it into its more active, cytotoxic form.

Enzymes that are useful include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs into free active drugs.

Other

Another type of covalent modification of the polypeptide comprises linking the polypeptide to one of a variety of nonproteinaceous polymers, e.g., polyethylene glycol, polypropylene glycol, polyoxyalkylenes, or copolymers of polyethylene glycol and polypropylene glycol. The polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 18th edition, Gennaro, A. R., Ed., (1990).

### Obtaining Polypeptides for Use in the Formulations

The polypeptides to be purified using reusable chromatography material cleaned by the methods described herein may be obtained using methods well-known in the art, including the recombination methods. The following sections provide guidance regarding these methods.

Polynucleotides

“Polynucleotide,” or “nucleic acid,” as used interchangeably herein, refer to polymers of nucleotides of any length, and include DNA and RNA.

Polynucleotides encoding polypeptides may be obtained from any source including, but not limited to, a cDNA library prepared from tissue believed to possess the polypeptide mRNA and to express it at a detectable level. Accordingly, polynucleotides encoding polypeptide can be conveniently obtained from a cDNA library prepared from human tissue. The polypeptide-encoding gene may also be obtained from a genomic library or by known synthetic procedures (e.g., automated nucleic acid synthesis).

For example, the polynucleotide may encode an entire immunoglobulin molecule chain, such as a light chain or a heavy chain. A complete heavy chain includes not only a heavy chain variable region (V_{H}) but also a heavy chain constant region (CH), which typically will comprise three constant domains: C_{H}1, C_{H}2 and C_{H}3; and a “hinge” region. In some situations, the presence of a constant region is desirable.

Other polypeptides which may be encoded by the polynucleotide include antigen-binding antibody fragments such as single domain antibodies (“dAbs”), Fv, scFv, Fab′ and F(ab′)₂ and “minibodies.” Minibodies are (typically) bivalent antibody fragments from which the C_{H}1 and C_{K} or C_{L} domain has been excised. As minibodies are smaller than conventional antibodies they should achieve better tissue penetration in clinical/diagnostic use, but being bivalent they should retain higher binding affinity than monovalent antibody fragments, such as dAbs. Accordingly, unless the context dictates otherwise, the term “antibody” as used herein encompasses not only whole antibody molecules but also antigen-binding antibody fragments of the type discussed above. Preferably each framework region present in the encoded polypeptide will comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions.

Suitably, the polynucleotides described herein may be isolated and/or purified. In some embodiments, the polynucleotides are isolated polynucleotides.

The term “isolated polynucleotide” is intended to indicate that the molecule is removed or separated from its normal or natural environment or has been produced in such a way that it is not present in its normal or natural environment. In some embodiments, the polynucleotides are purified polynucleotides. The term purified is intended to indicate that at least some contaminating molecules or substances have been removed.

Suitably, the polynucleotides are substantially purified, such that the relevant polynucleotides constitute the dominant (i.e., most abundant) polynucleotides present in a composition.

Expression of Polynucleotides

The description below relates primarily to production of polypeptides by culturing cells transformed or transfected with a vector containing polypeptide-encoding polynucleotides. It is, of course, contemplated that alternative methods, which are well known in the art, may be employed to prepare polypeptides. For instance, the appropriate amino acid sequence, or portions thereof, may be produced by direct peptide synthesis using solid-phase techniques (see, e.g., Stewart et al., Solid-Phase Peptide Synthesis W.H. Freeman Co., San Francisco, Calif. (1969); Merrifield, J. Am. Chem. Soc. 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the polypeptide may be chemically synthesized separately and combined using chemical or enzymatic methods to produce the desired polypeptide.

Polynucleotides as described herein are inserted into an expression vector(s) for production of the polypeptides. The term “control sequences” refers to DNA sequences necessary for the expression of an operably linked coding sequence in a particular host organism. The control sequences include, but are not limited to, promoters (e.g., naturally-associated or heterologous promoters), signal sequences, enhancer elements, and transcription termination sequences.

A polynucleotide is “operably linked” when it is placed into a functional relationship with another polynucleotide sequence. For example, nucleic acids for a presequence or secretory leader is operably linked to nucleic acids for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleic acid sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

For antibodies, the light and heavy chains can be cloned in the same or different expression vectors. The nucleic acid segments encoding immunoglobulin chains are operably linked to control sequences in the expression vector(s) that ensure the expression of immunoglobulin polypeptides.

The vectors containing the polynucleotide sequences (e.g., the variable heavy and/or variable light chain encoding sequences and optional expression control sequences) can be transferred into a host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection. For production of transgenic animals, transgenes can be microinjected into fertilized oocytes, or can be incorporated into the genome of embryonic stem cells, and the nuclei of such cells transferred into enucleated oocytes.

Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an Escherichia coli plasmid to a bacterium, such as of the genus Bacillus, then the transformation vector is sometimes called a “shuttle vector”. It may even be a construct capable of being transferred from an E. coli plasmid to an Agrobacterium to a plant.

Vectors may be transformed into a suitable host cell as described below to provide for expression of a polypeptide. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Construction of suitable vectors containing one or more of these components employs standard ligation techniques which are known to the skilled artisan.

The vectors may be for example, plasmid, virus or phage vectors provided with an origin of replication, optionally a promoter for the expression of the said polynucleotide and optionally a regulator of the promoter. Vectors may contain one or more selectable marker genes which are well known in the art.

These expression vectors are typically replicable in the host organisms either as episomes or as an integral part of the host chromosomal DNA.

Host Cells

The host cell may be a bacterium, a yeast or other fungal cell, insect cell, a plant cell, or a mammalian cell, for example.

A transgenic multicellular host organism which has been genetically manipulated may be used to produce a polypeptide. The organism may be, for example, a transgenic mammalian organism (e.g., a transgenic goat or mouse line).

Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P), Pseudomonas such as P. aeruginosa, and Streptomyces. These examples are illustrative rather than limiting. Strain W3110 is one particularly preferred host or parent host because it is a common host strain for recombinant polynucleotide product fermentations. Preferably, the host cell secretes minimal amounts of proteolytic enzymes. For example, strain W3110 may be modified to effect a genetic mutation in the genes encoding polypeptides endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan′; E. coli W3110 strain 37D6, which has the complete genotype tonA ptr3 phoA E15 (argF-lac)₁₆₉ degP ompT rbs7 ilvG kan′; E. coli W3110 strain 40B4, which is strain 37D6 with a non-kanamycin resistant degP deletion mutation; and an E. coli strain having mutant periplasmic protease. Alternatively, in vitro methods of cloning, e.g., PCR or other nucleic acid polymerase reactions, are suitable.

In these prokaryotic hosts, one can make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Eukaryotic microbes may be used for expression. Eukaryotic microbes such as filamentous fungi or yeast are suitable cloning or expression hosts for polypeptide-encoding vectors. Saccharomyces cerevisiae is a commonly used lower eukaryotic host microorganism. Others include Schizosaccharomyces pombe; Kluyveromyces hosts such as, e.g., K. lactis (MW98-8C, CBS683, CBS4574), K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans, and A. niger. Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the polypeptides as described herein and in some instances are preferred (See Winnacker, From Genes to Clones VCH Publishers, N.Y., N.Y. (1987). For some embodiments, eukaryotic cells may be preferred, because a number of suitable host cell lines capable of secreting heterologous polypeptides (e.g., intact immunoglobulins) have been developed in the art, and include CHO cell lines, various Cos cell lines, HeLa cells, preferably, myeloma cell lines, or transformed B-cells or hybridomas. In some embodiments, the mammalian host cell is a CHO cell.

In some embodiments, the host cell is a vertebrate host cell. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/−DHFR(CHO or CHO-DP-12 line); mouse sertoli cells; monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

### Articles of Manufacture

The polypeptides formulations described herein may be contained within an article of manufacture. The article of manufacture may comprise a container containing the polypeptide (e.g., a formulation of the polypeptide, a spray dried formulation of the polypeptide, or a controlled release form of the polypeptide). Preferably, the article of manufacture comprises: (a) a container comprising a composition comprising the polypeptide and/or the polypeptide formulation described herein within the container; and (b) a package insert with instructions for administering the formulation to a subject.

The article of manufacture comprises a container and a label or package insert on or associated with the container. Suitable containers include, for example, bottles, vials, syringes, etc. The containers may be formed from a variety of materials such as glass or plastic. The container holds or contains the formulation and may have a sterile access port (for example the container may be an intravenous solution bag or a vial having a stopper pierceable by a hypodermic injection needle). At least one active agent in the composition is the polypeptide. The label or package insert indicates that the composition's use in a subject with specific guidance regarding dosing amounts and intervals of polypeptide and any other drug being provided. The article of manufacture may further include other materials desirable from a commercial and user standpoint, including other buffers, diluents, filters, needles, and syringes. In some embodiments, the container is a syringe. In some embodiments, the syringe is further contained within an injection device. In some embodiments, the injection device is an autoinjector.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products.

### EXEMPLARY EMBODIMENTS

The invention provides the following exemplary embodiments.
1. A formulation comprising a polypeptide and a hydroxypropyl methylcellulose acetate succinate (HPMCAS).
2. The formulation of embodiment 1, wherein the HPMCAS comprises from about 8% to about 12% acetate and from about 7% to about 15% succinate.
3. The formulation of embodiment 2, wherein the HPMCAS comprises about 8% acetate and about 15% succinate (HPMCAS-LF) or about 12% acetate and about 7% succinate (HPMCAS-HF).
4. The formulation of any one of embodiments 1-3, wherein the ratio of the HPMCAS to protein in the formulation is from about 4:1 (mg/mg) to about 1:4 (mg/mg), optionally wherein the ratio is about 4:1 (mg/mg), about 1:1 (mg/mg), or about 1:4 (mg/mg).
5. The formulation of any one of embodiments 1-4, wherein the formulation further comprises a histidine buffer.
6. The formulation of embodiment 5, wherein the histidine buffer is a histidine HCl buffer.
7. The formulation of embodiment 5 or 6, wherein the histidine buffer is in the formulation at a concentration of about 5 mM to about 20 mM, optionally wherein the histidine buffer is in the formulation at a concentration of about 10 mM.
8. The formulation of any one of embodiments 1-7, wherein the formulation further comprises a polysorbate.
9. The formulation of embodiment 8, wherein the polysorbate is polysorbate 20. 10. The formulation of embodiment 8 or 9, wherein polysorbate in the formulation at a concentration of about 0.005% (w/v) to about 0.5% (w/v).
11. The formulation of any one of embodiments 8-10, wherein the polysorbate is in the formulation at a concentration of about 0.01% (w/v).
12. The formulation of any one of embodiments 1-11, wherein the pH of the formulation is about 5.5 to about 7.0.
13. The formulation of any one of embodiments 1-12, wherein the pH of the formulation is about 6.5.
14. The formulation of any one of embodiments 1-13, wherein the polypeptide is an antibody.
15. The formulation of any one of embodiments 1-14, wherein the polypeptide is a monoclonal antibody.
16. The formulation of any one of embodiments 1-15, wherein the polypeptide is a human antibody, a chimeric antibody or a humanized antibody.
17. The formulation of any one of embodiments 14-16, wherein the antibody is an antibody fragment selected from a Fab, Fab′-SH, Fv, scFv, and (Fab′)2 fragment, optionally wherein the antibody fragment is a (Fab′)2 fragment.
18. The formulation of any one of embodiments 1-17, wherein the polypeptide is in the formulation at a concentration of about 5 mg/mL to about 100 mg/mL.
19. The formulation of any one of embodiments 1-18, wherein the polypeptide is in the formulation at a concentration of about 10 mg/mL.
20. A formulation comprising:
an antibody at a concentration of about 5 mg/mL to about 50 mg/mL,
an HPMCAS-LF, wherein the ratio of the HPMCAS-LF to antibody in the formulation is about
4:1 (mg/mg), about 1:1 (mg/mg), or about 1:4 (mg/mg),
a histidine-HCl buffer at a concentration of about 5 mM to about 20 mM,
polysorbate 20 at a concentration of about 0.005% (w/v) to about 0.5% (w/v), and
wherein the pH of the formulation is about 5.5 to about 7.0.
21. A formulation comprising:
an antibody at a concentration of about 10 mg/mL,
an HPMCAS-LF, wherein the ratio of the HPMCAS-LF to antibody in the formulation is about
4:1 (mg/mg), about 1:1 (mg/mg), or about 1:4 (mg/mg),
a histidine-HCl buffer at a concentration of about 10 mM,
polysorbate 20 at a concentration of about 0.01% (w/v), and
wherein the pH of the formulation is about 6.5.
22. A formulation comprising:
an antibody at a concentration of about 5 mg/mL to about 50 mg/mL,
an HPMCAS-HF, wherein the ratio of the HPMCAS-HF to antibody in the formulation is about
4:1 (mg/mg), about 1:1 (mg/mg), or about 1:4 (mg/mg),
a histidine-HCl buffer at a concentration of about 5 mM to about 20 mM,
polysorbate 20 at a concentration of about 0.005% (w/v) to about 0.5% (w/v), and
wherein the pH of the formulation is about 5.5 to about 7.0.
23. A formulation comprising:
an antibody at a concentration of about 10 mg/mL,
an HPMCAS-HF, wherein the ratio of the HPMCAS-HF to antibody in the formulation is about
4:1 (mg/mg), about 1:1 (mg/mg), or about 1:4 (mg/mg),
a histidine-HCl buffer at a concentration of about 10 mM,
polysorbate 20 at a concentration of about 0.01% (w/v), and
wherein the pH of the formulation is about 6.5.
24. The formulation of any one of embodiments 20-23, wherein the antibody is a monoclonal antibody.
25. The formulation of any one of embodiments 20-24, wherein the antibody is a human antibody, a chimeric antibody or a humanized antibody.
26. The formulation of any one of embodiments 20-25, wherein the antibody is an antibody fragment selected from a Fab, Fab′-SH, Fv, scFv, and (Fab′)2 fragment, optionally wherein the antibody fragment is a (Fab′)2 fragment.
27. The formulation of any one of embodiments 1-26, wherein the formulation is spray dried to form a spray dried formulation of a polypeptide or antibody.
28. The spray dried formulation of a polypeptide or antibody of embodiment 27, wherein the polypeptide or antibody in the formulation is stable at about 90° C. for at least about 5 hours and/or is stable at about 37° C. for at least about 4 weeks.
29. The spray dried formulation of a polypeptide or antibody of embodiment 27 or 28, wherein the polypeptide or antibody in the formulation shows reduced aggregation and/or reduced chemical degradation.
30. The spray dried formulation a polypeptide or antibody of embodiment 29, wherein the reduced chemical degradation comprises reduced formation of succinimide variants of the polypeptide and/or reduced formation of pyroglutamate variants of the polypeptide.
31. The spray dried formulation a polypeptide or antibody of any one of embodiments 27-30, wherein the formulation is an amorphous glassy formulation.
32. The spray dried formulation of a polypeptide or antibody of any one of embodiments 27-31, wherein the spray dried formulation of a polypeptide or antibody is encapsulated in a polymer system that produces acidic microclimate.
33. The spray dried formulation of a polypeptide or antibody of any one of embodiments 27-32, wherein the spray dried formulation of a polypeptide or antibody is encapsulated in lactic acid/glycolic acid polymer system.
34. The spray dried formulation of a polypeptide or antibody of any one of embodiments 27-33, wherein the spray dried formulation of a polypeptide or antibody is encapsulated in poly(lactic-co-glycolic acid) (PLGA).
35. The spray dried formulation of a polypeptide or antibody of any one of embodiments 27-33, wherein the spray dried formulation of a polypeptide or antibody is encapsulated in a PLGA rod.
36. A composition comprising the formulation of any one of embodiments 1-27.
37. A composition comprising the spray dried formulation of a polypeptide or antibody of any one of embodiments 27-35.
38. A composition comprising a lactic acid/glycolic acid polymer particle comprising the formulation of any one of embodiments 1-27.
39. A composition comprising a lactic acid/glycolic acid polymer particle comprising the spray dried formulation of any one of embodiments 27-35.
40. The composition of embodiment 38 or 39, wherein the lactic acid/glycolic acid polymer particle is a PLGA particle.
41. The composition of any one of embodiments 38-40, wherein the lactic acid/glycolic acid polymer particle is a PLGA rod.
42. A method of preparing a spray dried polypeptide, the method comprising preparing a formulation of any one of embodiments 1-19, and subjecting the formulation to spray drying.
43. A method of preparing a spray dried antibody, the method comprising preparing a formulation of any one of embodiments 20-26, and subjecting the formulation to spray drying.
44. The method of embodiment 42 or 43, wherein the spray drying is performed using a spray dryer comprising an inlet and an outlet.
45. The method of embodiment 44, wherein the inlet has a temperature of about 90° C. to about 120° C., about 100° C., or about 110° C.
46. The method of embodiment 44, wherein the outlet has a temperature of about 60° C.
47. A method of preparing a lactic acid/glycolic acid polymer particle comprising a polypeptide, the method comprising encapsulating the formulation of any one of embodiments 1-26 in a lactic acid/glycolic acid polymer system.
48. A method of preparing a lactic acid/glycolic acid polymer particle comprising a polypeptide, the method comprising encapsulating the spray dried formulation of any one of embodiments 27-30 in a lactic acid/glycolic acid polymer system.
49. The method of embodiment 47 or 48, wherein the lactic acid/glycolic acid polymer particle is a PLGA particle.
50. An article of manufacture comprising the formulation of any one of embodiments 1-27, the spray dried formulation of any one of embodiments 28-35, or the composition of any one of embodiments 36-41.
51. An article of manufacture comprising spray dried formulation prepared by the method of any one of embodiments 42-46.
52. An article of manufacture comprising lactic acid/glycolic acid polymer particle encapsulated polypeptide prepared by the method of any one of embodiments 47-49.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention are illustrated by the following non-limiting Examples. The disclosures of all references in the specification are expressly incorporated herein by reference.

### EXAMPLES

Abbreviations: AS-HF (HPMCAS with ˜12% acetate and ˜7% succinate); AS-LF (HPMCAS with ˜8% acetate and ˜15% succinate); E/P (excipient/protein ratio); HPMC (hydroxypropyl methyl cellulose, also known as hypromellose); HPMCAS (hypromellose acetate succinate); IEC (ion-exchange chromatography); PLGA (poly(lactic-co-glycolic acid)); and SEC (size-exclusion chromatography).

The examples below are intended to be purely exemplary of the invention and should therefore not be considered to limit the invention in any way. The following examples and detailed descriptions are offered by way of illustration and not by way of limitation.

### Example 1. Hydroxypropyl Methyl Cellulose Derivatives Stabilize Antibody Fragments and Resist pH Changes

The utility of acetate and succinate derivatives of hydroxypropyl methyl cellulose (HPMC or Hypromellose) in stabilizing a model protein (labeled Fab2) in the context of PLGA rods was evaluated. Two different HPMC derivatives were investigated; one with ˜8% acetate and ˜15% succinate (referred to as AS-LF) and the other with ˜12% acetate and 7% succinate acid (referred to as AS-HF).

The performance of HPMCAS excipients in spray dried formulations with Fab2 was compared to that of trehalose, metolose and an excipient devoid formulation at 90° C. for a few hours and at 37° C. for a duration of 4 weeks. Trehalose has been studied previously for stabilization of Fab2 in spray dried formulations and at elevated temperatures (Rajagopal, K et al., Mol Pharm, 2019. 16(1):349-358). A temperature of 90° C. was selected to guide the compatibility of spray dried formulation for high temperature processing such as hot-melt extrusion used in the preparation of PLGA polymer rods for drug delivery. Stability at 37° C. was performed to provide meaningful relative stability between the formulations that could be applicable at 2-8° C. In addition, 37° C. also mimics the bodily temperature and captures the effect of exposing the protein drug to physiological temperature experienced during long-term residence within a sustained delivery system of PLGA rod. Fab2, as a spray dried powder, was encapsulated within PLGA cylindrical rods by a hot melt extrusion method. Following which Fab2 stability inside PLGA rod when formulated in presence of HPMCAS excipients, metolose and trehalose under physiological condition was studied at 37° C. for a duration of 3 weeks. To characterize protein stability upon encapsulation in PLGA rods, Fab2 extracted from rods were examined for aggregation or chemical degradation. Although unlike trehalose which is hydrolyzable in humans, HPMCAS is non-biodegradable and hence applications in parenteral delivery are limited.

### Materials and Methods

Materials. Fab2 was obtained from Genentech (South San Francisco, CA). α-α Trehalose dihydrate was obtained from Ferro Pfanstiehl Laboratories (Cleveland, OH), Hypromellose Acetate Succinate (HPMCAS) was obtained from Shin-Etsu Chemical Co., Ltd. (Tokyo, Japan) in two different grades HPMC AS-HF (12% Acetyl, 7% Succinoyl, dissolving pH≥6.5) and HPMC AS-LF (8% Acetyl, 15% Succinoyl, dissolving pH≥5.5). Hypromellose (Metolose) was also obtained from Shin-Etsu Chemical Co., Ltd. (Tokyo, Japan). Histidine and histidine-HCl were obtained from Sigma-Aldrich (St. Louis, MO), and polysorbate 20 (PS20) was obtained from Spectrum Chemical (New Burnswick, NJ). PLGA RESOMER® RG755S was purchased from Evonik Corporation (Piscataway, NJ).

Spray Drying of Fab2. Fab2 at 40 mg/mL was dialyzed (MWCO=10,000 Da) against 10 mM histidine/histidine-HCl buffer (pH 5.5) containing 0.01% (w/v) polysorbate 20. The dialysis buffer was changed four times in 16 hours. Fab2 was diluted to 20 mg/mL with 10 mM histidine/histidine-HCl buffer (pH 5.5) containing 0.01% polysorbate 20. UV spectroscopy was used to measure Fab2 concentration (ε=1.39 mL·cm-1·mg-1 at 280 nm). As specified by manufacturer HPMC AS-LF and HPMC AS-HF grades were insoluble in water initially when prepared at 20 mg/mL with a final pH˜4 of the mixture. Working solutions of HPMC AS-LF and HPMC AS-HF at 20 mg/mL were prepared in water containing 10 mg/mL histidine base and 0.01% polysorbate 20 at a final pH of 6.5. The required amount of trehalose dihydrate, HPMC AS-LF, HPMC AS-HF and Metolose were added to Fab2 resulting in solutions with the same excipient/protein mass ratio of 1.0 (see Table 1). Then, aqueous formulations were spray-dried using Buchi Model B290 Mini spray dryer (New Castle, NJ) at an inlet and outlet temperature of 110±2 and 60±2° C., respectively. The pump power was 8%, and the aspirator was operated at 100% capacity. The liquid feed rate was 3.4 mL/min, and the compressed air flow rate was 600 L/h. The collected spray-dried powders were transferred to a clean, dry 15 cc Lyo glass vial and stored under nitrogen in a vacuum chamber until further use. All spray dried powders were further secondary dried in a lyophilizer (Advantage Pro Lyophilizer, SP Scientific, UK).

Scanning Electron Microscopy (SEM). The morphology of the spray-dried particles was imaged with a Quanta 3D (Hillsboro, OR) FEG SEM. The samples were fixed on aluminum stubs with carbon adhesive tape and sputter coated with gold/palladium (Cressington Sputter Coater, TED Pella, Inc.) to improve their electrical conductivity. SEM images were collected at a low voltage to minimize any potential sample damage or surface charging.

Particle Size Characterization by Laser Diffractions. The particle size distributions of spray-dried Fab2 powders were measured using a Partica LA-950V2 Laser Diffraction Particle Size Distribution Analyzer (Horiba Ltd., Kyoto, Japan). Approximately 1 mg of spray-dried powder was dispersed in 1 ml of isopropanol and the dispersion was added dropwise to 50 ml isopropanol until a target light obscuration level was achieved. The refractive index of isopropanol (1.3776) was used to calculate the size distribution using the particle sizing program.

High Temperature Heat Treatment of Fab2. Spray-dried powder (2-7 mg, equivalent to 1 to 1.5 mg Fab2) from each formulation was weighed into 7 mL glass vials. The vials were uncapped and placed in a Binder forced-air convection oven (Bohemia, NY), preheated, and equilibrated to the desired temperature at 90±5° C. Samples were removed at 1, 2, 3, 4, and 5 h, capped immediately, and allowed to cool to room temperature. The vial contents were dissolved in purified water such that the final protein concentration was ˜1 mg/mL. The reconstituted aqueous samples were observed for clarity, and visibly clear samples were used for SEC and IEC analysis.

Size Exclusion Chromatography (SEC). For Fab2 Size-exclusion chromatography (SEC) HPLC was performed using an Agilent 1200 series HPLC system (Santa Clara, CA) equipped with a TOSOH TSKgel G2000SWXL (7.8×300 mm i.d., 5 μm particle size) column. Samples were analyzed at 25° C. in isocratic mode with 0.20 M K3PO4 and 0.25 M KCl, pH 6.2 as mobile phase at a flow rate of 0.7 mL/min. A 20 μL sample at 1 mg/mL concentration was injected, and the total run time was 20 min. Absorbance at 280 nm was used for detection. The percent peak area was calculated by dividing the peak area of each group at each time point to the total peak area.

Ion-Exchange Chromatography (IEC). Ion-exchange chromatography (IEC) HPLC was performed using an Agilent 1200 series HPLC system (Santa Clara, CA) on two Dionex (Sunnyvale, CA) ProPac SAX-10 (2×250 mm) strong anion-exchange columns connected in series and equipped with a diode array detector (DAD). Mobile phase A (solvent A) was 20 mM Tris buffer, pH 8.2, and mobile phase B (solvent B) was 250 mM sodium chloride dissolved in solvent A. Prior to analysis, the samples were diluted to approximately 1 mg/mL in solvent A, and 20 μL of sample was injected. A linear gradient starting from 100% solvent A at 0 min to 20% solvent A at 45 min was employed to separate Fab2 charge variants in a total of ˜60 min run time. Absorbance at 280 nm was used for detection. The IEC peaks were separated into a main peak, acidic peak, and basic peak.

Separation of Fab2 from AS-LF and AS-HF spray dried formulations. Stability of Fab2 in the HPMCAS powder formulations was measured analytically via SEC and IEC by reconstituting the powder in water and separating Fab2 from HPMCAS. The reconstituted Fab2 spray dried powders with AS-LF and AS-HF were titrated with 0.1 N HCl to bring the pH of solution down to pH 3 and precipitate out the HPMCAS excipient. The precipitated sample was centrifuged at 4000 g and the supernatant was used for Fab2 analysis purposes by diluting in a mobile phase corresponding to the assay.

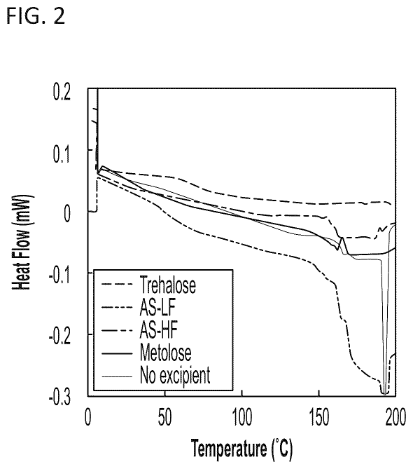

Differential Scanning calorimetry (DSC). DSC was performed on a TA instrument DSC Q200 (New Castle DE) calorimeter under the modulation condition. Approximately 2±1 mg sample was weighed in an aluminum pan and hermetically sealed. The sample was equilibrated at 5° C. for 10 min and then heated at 2° C./min with ±1.00° C./min modulation to 120° C. and cooled back to 5° C. at 2° C./min. After the sample was equilibrated again at 5° C., the same heating and modulation ramp was repeated for a second time all the way to 180° C. The first heating ramp was done to eliminate any thermal history associated with the sample because of storage or handling conditions. For reporting purposes, the second heating ramp of the sample was used, which is free from any thermal history. The glass transition temperature was reported as the half-height midpoint of the second heating cycle (FIG. 2).

Base and Acid Titrations. Base titration experiments were carried out on separate solutions of HPMC AS-LF and HPMC AS-HF excipients in water using a Mettler Toledo pH meter (Mettler Toledo, Columbus, OH) with a Micro-Pro-ISM pH probe. Mixtures of HPMC AS-LF and HPMC AS-HF in water were prepared at 5% and 7% by weight respectively. Initial pH of both the mixtures were measured to be around 4 and majority of HPMCAS excipients were insoluble in water. The solutions were gradually titrated with 0.5N NaOH and with continuous swirling until completely miscible. The pH of both HPMC AS-LF and HPMC AS-HF in water measured to be in excess of pH 6 upon complete miscibility. Similar acid titration experiments were carried out for spray dried powder samples of HPMCAS, metolose, trehalose and no excipient formulation in water, in this case by titrating down to pH 3 with 0.1N HCl.

Hot Melt Extrusion (HME) of spray dried Fab2 and PLGA cylindrical rods. Hot-melt extrusion was performed using a HAAKE™ MiniCTW Micro-Conical Twin Screw Compounder (Thermo Scientific, Karlsruhe, Germany) with a barrel capacity of 5 g or 7 cm3. The extruder barrel was pre-heated to 90° C. and screw speed was set to 40 rpm. The front barrel opening of the extruder was kept closed in the recirculation mode. The pre-weighed solid polymer and spray dried Fab2 were manually fed slowly into the extruder and re-circulated back to the barrel for micro-compounding. The Fab2 loading for implants using different spray dried formulations was fixed at 15% (w/w) Fab2/PLGA. The melt-phase blending was continued for 30 min under recirculation mode after complete feeding and then extruded through a 0.5 mm circular die. The extrudates were cooled to room temperature, cut to cylinders of desired length and stored at 2-8° C. until further use in release studies.

In vitro Stability Study Setup. In vitro stability setup for studying Fab2 from PLGA rods was performed in glass vials (VWR borosilicate glass vials, 7.4 mL) with 5 mg of polymer rod and 1 mL of PBSTN (1×PBS with 0.01% (w/v) PS20 and 0.02% (w/v) sodium azide). Study temperature was maintained at 37° C. in a humidity-controlled incubator at 80% RH to minimize evaporation. For sample preparation, the polymer rods were cut into five 1 mg segments and placed in individually labeled glass vials. The exact weight of the rod was recorded using a micro balance (Mettler Toledo XPE56, Columbus, OH). The release study was done in triplicate and the release buffer was sampled on day 1 and then every 7 days. At every time point, the release buffer (PBSTN) was completely pipetted out and collected in 1.5 mL LoBind® Eppendorf tubes (Eppendorf, CT, USA) and replaced with 1 mL of fresh PBSTN. The Fab2 concentration in the release buffer was measured using the SEC method as described previously. After 3 weeks of release the buffer was thoroughly aspirated off the rods in vial, then the rods were rinsed twice with 1 mL water and were allowed to air dry for 3-4 hours inside a chemical/biosafety cabinet until there was no visible liquid in the vial. The rods were then placed in a vacuum desiccator for at least a week to completely dry out all the water.

Recovery of Fab2 from PLGA rods. After drying out the rods, the same rods were used for analyzing Fab2 remaining in the rods. An extraction protocol was used to remove and isolate Fab2 from the polymer. First, the dry rods were transferred to tapered bottom glass vials (Worldwide Glass Resource, 4 mL), then the rods were dissolved by adding 4 mL tetrahydrofuran (THF) and agitating the vials until the rods were visibly dissolved. PLGA is soluble in THF and proteins are not. The vials with dissolved PLGA rods were centrifuged to create a Fab2 pellet at the bottom of the vial. The protein pellet is easily disturbed, so approximately 3.5 mL of the THF was removed from the supernatant. The vials were then rinsed an additional time with THF and centrifuged. The Fab2 pellet at the bottom of the tube was rinsed a second time with THF and centrifuged to repeat the process again. This was done to remove any remaining polymer residues post first rinse with THF. Thus, leaving a Fab2 rich pellet in wet powder form. The remaining THF was evaporated off leaving dried Fab2 powder. The final dried Fab2 powder was reconstituted with 1 ml of PBS buffer and used for analytical testing.

### Results

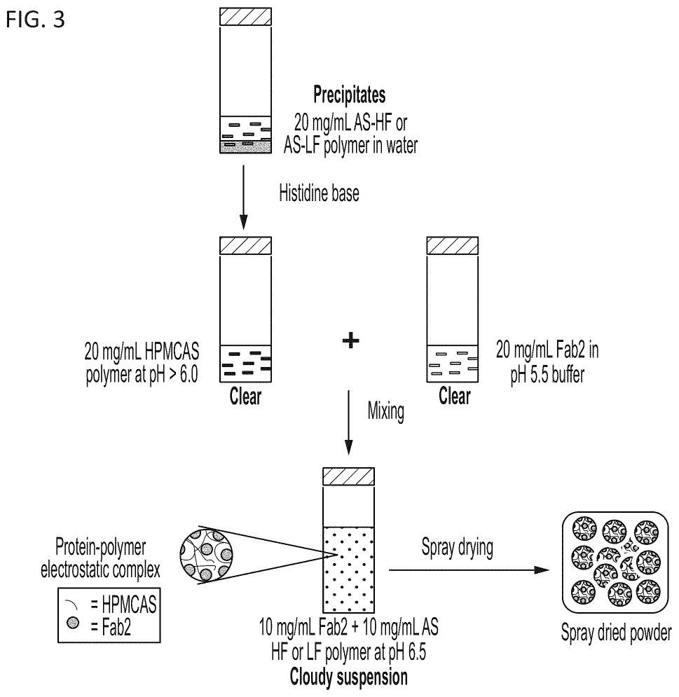

Spray Dried Formulations of Fab2. Five formulations of Fab2 at 10 mg/mL were prepared and evaluated for stability after spray drying (Table 1-1). All of the five formulations were formulated in 10 mM His-HCl/His buffer and 0.01% w/v polysorbate 20. One of the formulations had only 10 mM His-HCl/His buffer and 0.01% w/v polysorbate20 without any sugar or HPMCAS which was used as control. The two formulations with HPMCAS excipients were formulated at pH 6.5, 10 mM His-HCl/His buffer and 0.01% w/v polysorbate 20. The preparations of spray dried AS-LF and AS-HF formulations with Fab2 is shown schematically in FIG. 3. The mass ratio of trehalose, metolose, AS-LF and AS-HF excipient to protein in the respective formulations was 1:1. The molar ratio of excipient to protein however is different due to differences in the excipient molecular mass and ranged between 0.3 to 137 depending on the formulation. The pH of all formulations were measured before spray drying in liquid state as well as after spray drying by reconstitution in water as shown in Table 1-1. Both the pH measurements were identical indicating no change in ionization state due to spray drying.

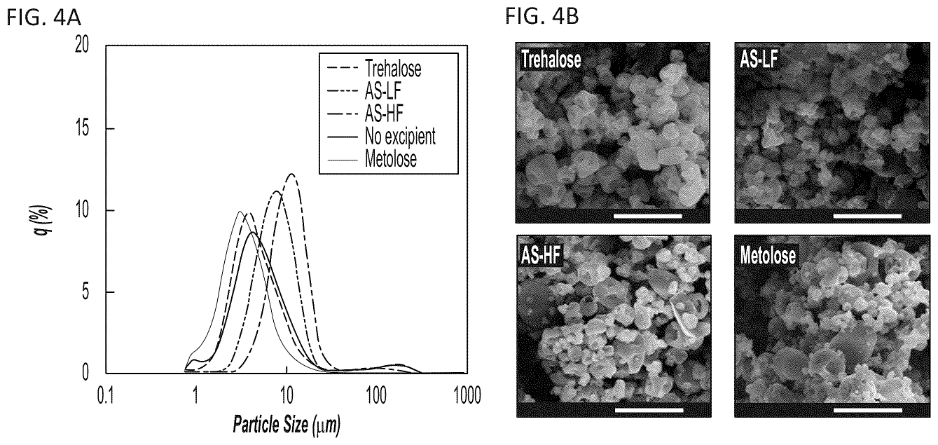

Properties of Spray Dried Fab2. All five formulations after spray drying yielded micron-sized particles (FIG. 4A) with similar particle morphology ranging from spherical to collapsed spheres (FIG. 4B). The particles were ranging from 3 to 10 microns in diameter across the different excipient formulations. All spray dried powders were reconstituted in water and analyzed for aggregation using size-exclusion chromatography (SEC) and for chemical degradation using ion-exchange chromatography (IEC). For formulations with AS-LF and AS-HF a separation protocol was followed as highlighted in the methods section before analyzing Fab2. The monomer content by SEC and the major charge variant peak by IEC were similar for all formulations after spray drying and comparable to Fab2 before spray drying (Table 1-1).

TABLE 1-1
Details of spray dried Fab2 formulations evaluated for stability studies

| | | | | | Stability after | | | |
|---|---|---|---|---|---|---|---|---|
| | | | Spray dried | | spray drying | | | |
| | | | powder | | | Main | | |
| | Excipient/Protein | | Particle | | Monomer | Peak | pH | |
| | ratio | | size | T_{g} | SEC | IEC | Before | After |
| Excipient | mg/mg | mole/mole | μm | ° C. | % | % | spraying | spraying¹ |
| No | 0 | 0 | 9.2 | NA | 99.4 | 82.6 | 5.4 | 5.4 |
| excipient | | | | | | | | |
| Trehalose | 1 | 137 | 5.2 | 78.8 | 99.8 | 83.0 | 5.5 | 5.5 |
| Metolose | 1 | 0.3 | 3.0 | 150.2 | 99.6 | 82.5 | 5.4 | 5.4 |
| AS-LF | 1 | 0.5 | 5.9 | 58.6 | 99.8 | 82.6 | 6.5 | 6.5 |
| AS-HF | 1 | 0.4 | 9.8 | 67.3 | 99.8 | 80.6 | 6.7 | 6.7 |

¹pH after spray drying was measured after dissolving 1% solid in water.

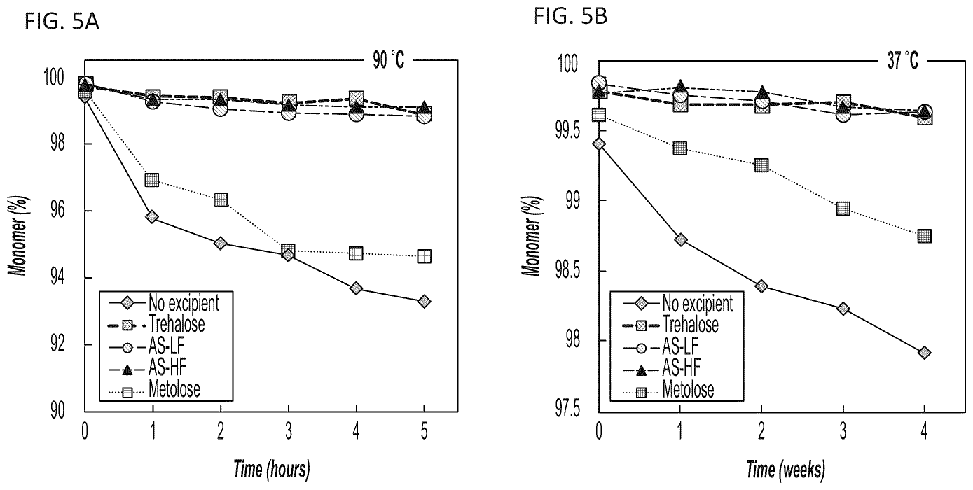

Effect of HPMCAS on Solid-State Stability of Fab2. The stability of Fab2 in spray dried formulations was tested at 90° C. for 5 hours and at 37° C. for 4 weeks. Maximum level of aggregation was observed in case of the formulation devoid of excipient followed by metolose containing formulation at both temperatures. At 90° C. maximum monomer loss was 6.2% after 5 hours (FIG. 5A) and at 37° C. monomer loss was 1.5% after four weeks (FIG. 5B) in case of excipient devoid formulation. Followed by metolose formulation with 5% monomer loss at 90° C. and 0.9% monomer loss at 37° C. In formulations containing HPMCAS excipients and trehalose, monomer loss was comparable and significantly reduced irrespective of the nature of the excipient. At 90° C. incubation for five hours, the trehalose, AS-LF and AS-HF formulations showed the least monomer loss (0.7%-0.9%). At 37° C. incubation for four weeks, the trehalose, AS-LF and AS-HF formulations again showed the least monomer loss (<0.2%). Shorter incubation period at 90° C. caused more aggregation than longer incubation period at 37° C. In comparison of the excipient-dependent physical aggregation of Fab2 upon storage at both temperatures, polymeric excipients AS-LF and AS-HF provided equal protection as trehalose against aggregation.

The IEC method relied on an anion exchange column for separating the different Fab2 related charge variant species into either basic or acidic variants. Basic peaks were observed to the left of the Fab2 main peak and acidic charge variants appeared on the right of the Fab2 main peak. Detailed characterization and identity of the peaks on IEC and chemical degradation mechanism of Fab2 has been reported previously (Rajagopal, K et al., Mol Pharm, 2019. 16(1):349-358). The peak immediately to the left of Fab2 main peak has been studied as pyroglutamate peak formation that is formed via the intramolecular cyclization of N-terminal glutamic acid in Fab2. The peaks around 19 and 21 minutes correspond to succinimide intermediate products of two and one aspartic acid residues in the Fab2 sequence respectively. Such formations of pyroglutamate and succinimide intermediate products lead to a net negative charge removal and gives rise to basic peaks on anionic IEC. Whereas, formation of deamidation species from asparagine side chain results in a net negative charge and increase in Fab2 isoelectric point (pI), thereby leading to acidic variant peaks on anionic IEC. No major Fab2 deamidation peaks were observed during assaying.

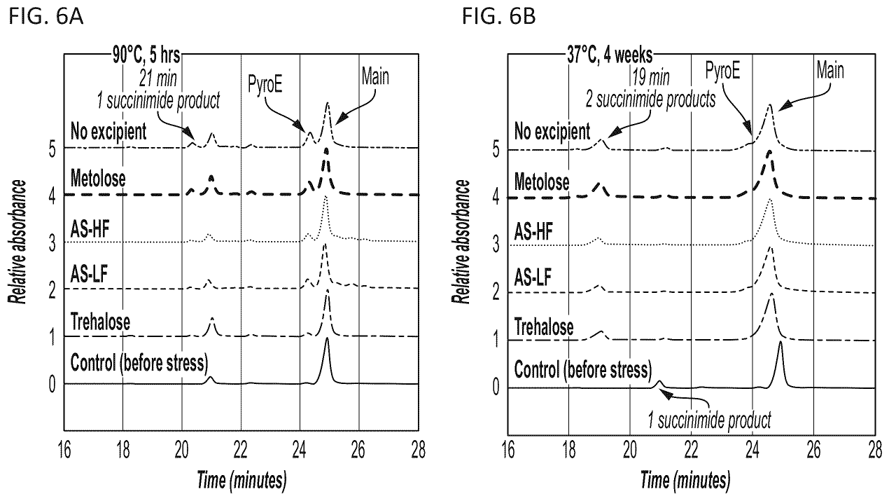

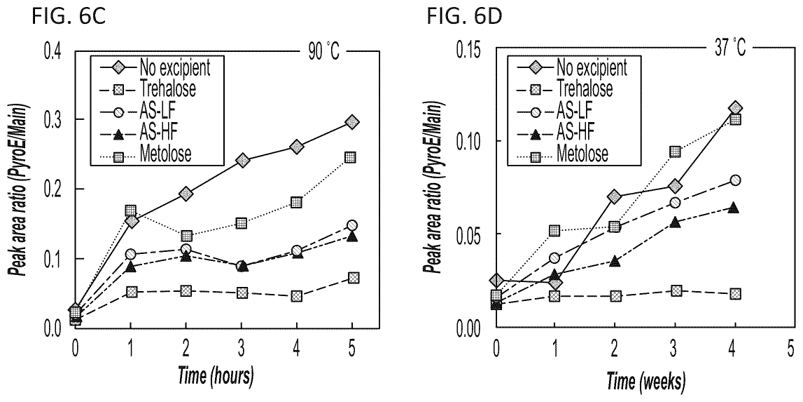

Chemical degradation was observed in case of Fab2 upon exposure to 90° C. and 37° C. with varying pathways (FIGS. 6A and 6B). The succinimide product formed via the cyclization of one aspartic acid side chain in Fab2 and pyroglutamate product (PyroE) formed via the cyclization of N-terminal glutamic acid gave rise to basic peaks around 21 minutes and 24.2 minutes respectively on IEC. An increase in both these degradation reactions were seen upon exposing Fab2 to 90° C. for all formulations (FIG. 6A). Whereas at 37° C. a new peak appeared around 19 minutes in all formulations after stress (FIG. 6B) and the peak around 21 minutes disappeared. Suggesting the formation of a new succinimide product from a second aspartic acid after exposure to 37° C.

Both, at 90° C. and 37° C. the presence of excipient in the formulation did not influence succinimide formation. However, the nature of the excipient showed to have an effect on the magnitude of pyroglutamate formation. The IEC chromatograms have been normalized with respect to the main peak. Pyroglutamate formation was quantified by measuring the ratio of main peak and pyroglutamate peak areas (FIGS. 6C and 6D). The presence of HPMCAS excipients didn't have any effect on Fab2 pyroglutamate formation, and in contrast to trehalose which suppressed pyroglutamate peak formation compared to an excipient devoid formulation. This coincides with our previous results that trehalose suppressed pyroglutamate peak formation in Fab2 under similar conditions (Rajagopal, K et al., Mol Pharm, 2019. 16(1):349-358). Concurrently, the presence of HPMCAS excipients didn't improve Fab2 chemical stability during both 90° C. and 37° C. stress conditions.

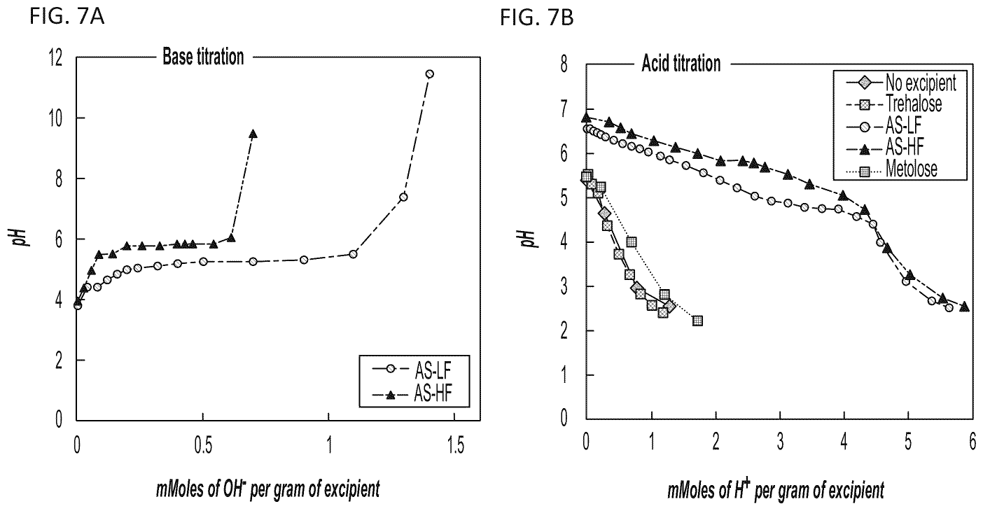

Buffering Capacity of HPMCAS Excipients. Base titration experiments of HPMCAS raw materials revealed the resistance of both excipients to pH change upon addition of sodium hydroxide (FIG. 7A). Such base titrations mimicked similar aqueous surroundings by HPMCAS chains during preparation of HPMCAS stock solution for spray drying (FIG. 3). AS-LF showed resistance to a change in pH around pH 5.2 and AS-HF around pH 5.8 upon subsequent base addition further confirming the fact that these two excipients did provide the range of buffering capacity as specified by the manufacturer. During the base titration experiments, AS-LF was more resistant to pH change within its observed buffering range around pH 5.2 i.e. almost twice that of AS-HF as determined from mMoles of hydroxyl groups introduced per mass of each excipient (FIG. 7A). To examine the neutralization effect of HPMCAS excipients inside an acidic microclimate of PLGA device, change in pH was monitored upon acid titration of HPMCAS spray dried powder formulations with strong hydrochloric acid (FIG. 7B). Presence of HPMCAS excipient in the spray dried Fab2 powder resisted the pH change with addition of acid around pH 5. Upon comparison with spray dried protein powders without HPMCAS excipients the pH drop in case of trehalose and no excipient formulation was significant (from pH 5.5-2.5) with addition of just 1 mM of acid. Similarly, in case of metolose which is structurally identical to HPMCAS minus the acetate and succinate functional groups, no buffering capacity was seen. Importantly, it was interesting to find that for HPMCAS containing spray dried protein formulations to affect a similar change in pH almost 6 times higher (≈6 mM) equivalent amount of the acid was required as compared to formulations without HPMCAS.

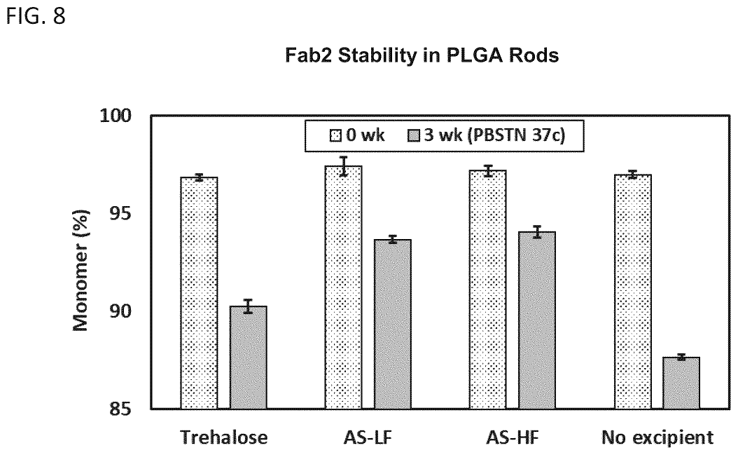

Stabilization of Fab2 Encapsulated in PLGA Implant During In Vitro Release. To examine the effect of excipients on protein stability within a PLGA implant rod, spray dried Fab2 formulations with Trehalose, AS-LF, AS-HF and without any excipient were encapsulated within a 1 mg PLGA cylindrical rod for studying drug release and PLGA implant degradation. Recovered Fab2 from 15% drug loaded PLGA rods at 37° C., after 3 weeks incubation in PBSTN was analyzed for aggregate stability (FIG. 8). This was done in triplicate for the different Fab2 loaded PLGA rods. In the absence of any excipient, Fab2 aggregation was higher and the monomer content decreased by 9.5%. Trehalose containing rod formulation showed a similar loss in monomer content of 7% after 3 weeks. For all the HPMCAS excipient formulations, the least amount of Fab2 aggregates were observed with monomer content decreasing by 4% (AS-LF) and 3.2% (AS-HF).

### DISCUSSION

Spray drying provides a physically stable amorphous drug form that enables processing of the spray dried product into poorly soluble polymeric drug delivery systems. The amorphous drug can be molecularly dispersed in a stable micron form for sustained delivery drug release systems like millimeter scale PLGA cylindrical rods. The thermogravimetric analysis of the spray dried powder (FIG. 2) and glass transition temperature (Tg) measurements (Table 1-1) can provide information on identifying the hot melt extrusion processing temperature window for preparation of PLGA rods. Also, spray drying produces a solid drug form of uniform size and morphology that can be manufactured via a reproducible, controllable, and scalable process. The physical characterization of the spray dried powder suggests that the nature of the excipient doesn't affect the overall size or morphology of the spray dried powder as evidenced from the SEM images (FIG. 4). The collapsed shape of our particles have been reported before in similar drying experiments which is a function of formulation composition and spray drying parameters like temperature, air flow and feed rate (Rajagopal, K et al., Mol Pharm, 2019. 16(1):349-358).

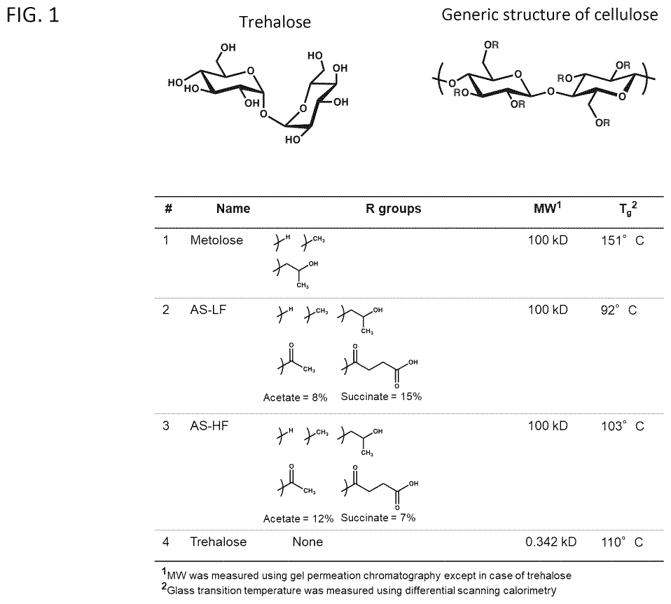

Controlling the spray drying conditions is important for maintaining protein stability during the entire operation and for further processing. The stability of our spray dried Fab2 seemed to be unaffected by drying conditions as well as the nature or absence of excipient. Moreover, since drying is a rapid process of solvent evaporation and droplet formation, having the right amount of excipient is crucial for a homogenous protein-excipient solid phase matrix with one single Tg value. Since, the property of dispersion is related to Tg of the pure excipient and final relative humidity (RH) of the product (Jang, J W et al., PDA J Pharm Sci Technol, 1995, 49(4):166-74). The type of excipient affected T_{g} of the spray dried protein powder. Concomitantly, the powder T_{g} corresponded with the pure excipient T_{g} (FIG. 1), and greater the T_{g} of excipient, the higher the T_{g} of overall excipient-protein formulation. Hence, the T_{g} of Trehalose formulation (79° C.) is higher than AS-HF (67° C.) and AS-LF formulations (59° C.). Since, all the spray dried formulations were processed under similar RH condition, the effect of moisture should be of minimal significance.

Previous studies have demonstrated that Fab2 is known to undergo aggregation and chemical degradation when stressed at 90° C. in the absence of excipient (Rajagopal, K et al., Mol Pharm, 2019. 16(1):349-358). In the presence of AS-LF or AS-HF, we have confirmed that Fab2 is stable in powder form when exposed to 90° C. and 37° C. providing similar protection as trehalose against aggregation (FIGS. 5A and 5B). However, presence of HPMCAS did not suppress pyroglutamate formation in solid-state formulation (FIG. 6A-6D). As the nature of excipient would determine the right amount of excipient loading required for protection of an antibody (Costantino, H R et al., J Pharm Sci, 1998, 87(11):1412-20) further optimization of excipient loading to probe the maximum protection provided by HPMCAS is necessary. However, HPMCAS as a polymeric excipient might be superior to a small molecule excipient trehalose in preferentially protecting antibodies upon storage inside the high humidity condition of a PLGA rod, and during drug release due to its slower global mobility Mensink, M A et al., Eur J Pharm Biopharm, 2017, 114:288-295). The present studies have confirmed that at an excipient to protein mass ratio of 1.0, HPMCAS excipients provide superior protection to Fab2 inside a PLGA rod under release conditions.

The solid state stabilization of proteins by a good excipient can be explained on the basis of: its glass transition, tendency for water replacement, and its chemical stability (Colaco, C J et al., Science, 1995, 268(5212):788). In the dry state, the excipient of choice should be able to prevent protein denaturation by preferential exclusion of water molecules. One such excipient of choice is trehalose, which provides maximum hydrogen bonding protection to proteins in the dry state (Allison, S D et al., Arch Biochem Biophys, 1999, 365(2):289-98). Thus, replacement of water molecules and direct protein-excipient interaction through hydrogen bonding are benchmarks to maintain protein in a folded state in the solid formulation. The saturation concentration of trehalose for providing maximum protection to Fab2 has been determined at an excipient to protein mass ratio of 1.0 in our previous studies (Rajagopal, K et al., Mol Pharm, 2019. 16(1):349-358). At the current excipient to protein mass ratio of 1.0, the moles of HPMCAS excipient is much lower as compared to that of trehalose. It has been reported that polymeric sugars hydrogen bond differently with proteins than disaccharides (Allison, S D et al., Arch Biochem Biophys, 1999, 365(2):289-98). Although probing the strength of hydrogen bonding interaction between Fab2 and HPMCAS is out of our current scope, HPMCAS seems to provide equivalent protection to Fab2 against aggregation at 37° C. and 90° C. similar to trehalose (FIGS. 5A and 5B). Further studies would be necessary to determine the concentration dependence of Fab2 stability on HPMCAS.

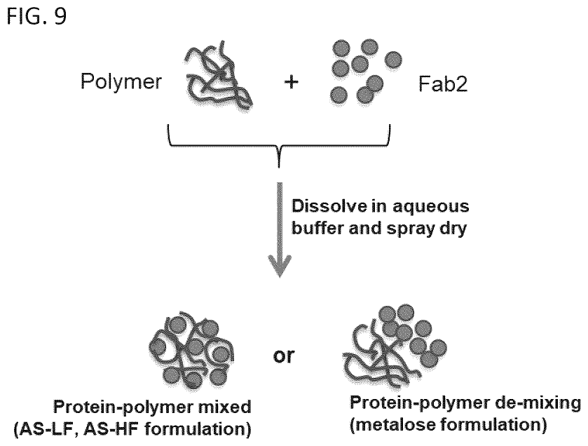

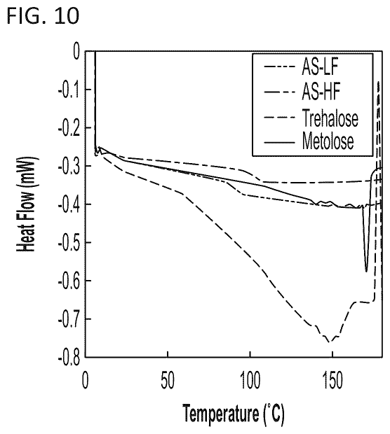

The data presented here support using HPMCAS as an amorphous solid dispersion carrier for protein drugs at high temperature. The physical stability of HPMCAS formulation is comparable to that of trehalose at 90° C., which could be an effect of the type and strength of hydrogen bonding with protein in a glassy matrix. The hydroxyl groups of HPMCAS can form hydrogen bond with protein and extend stability of the spray dried solid. This should restrict any molecular mobility, phase separation with formation of a tighter protein-excipient interaction existing in a proper protein-polymer mixed phase (FIG. 9). Al-Obaidi et al. have reported HPMCAS as a strong hydrogen bond donor, improves the amorphous phase stability of a hydrophobic drug via hydrogen bonding interaction with the drug carbonyl group (Al-Obaidi, H. and G. Buckton, AAPS PharmSciTech, 2009, 10(4):1172-7). In addition, presence of a succinate moiety facilitates additional intermolecular hydrogen bonding interaction between HPMCAS and protein. The free carboxylic acid group in succinate could act as both hydrogen bond donor and acceptor to protein amide group. Concurrently metolose with similar methoxy and hydroxypropoxy groups as HPMCAS but devoid of an additional succinate group would be deficit of a hydrogen bond donor and acceptor site for the protein. Hence, the protein excipient interaction would be weaker in case of a metolose formulation. As evidenced from the Tg (150° C.) value of the metolose spray dried formulation it likely exists as separate protein and excipient phases in the dried state (FIG. 9) driven by protein-polymer demixing. Thus, metolose spray dried has the same Tg value as that of pure metolose (FIG. 1 and FIG. 10), unaffected by any protein interaction. On the contrary, a spray dried molecular dispersion of protein and HPMCAS produced one single amorphous phase with different glass transition than of pure HPMCAS (Table 1-1 and FIG. 10).

It is interesting that trehalose and not HPMCAS prevents chemical degradation of Fab2 in the solid state (FIGS. 6C and 6D). All spray dried formulations had an increase in basic variants upon stress at 90° C. with the least amount of basic variants being formed in trehalose containing formulation (FIGS. 6A and 6B). This has been reported previously in case of Fab2 as it is prone to heating dependent dehydration/isomerization that contribute to decreased chemical stability (Rajagopal, K et al., Mol Pharm, 2019. 16(1):349-358). It is noteworthy that succinimide and pyroglutamate formation is more in HPMCAS formulation than in trehalose containing formulation. Pyroglutamate is a product of cyclization of N-terminal glutamic acid on protein. As hypothesized in our previous studies hydrogen bonding interaction between donor hydroxyl group of trehalose and acceptor N-terminal amine group of Fab2 could suppress pyroglutamate formation (Rajagopal, K et al., Mol Pharm, 2019. 16(1):349-358). Surprisingly, a similar amine-hydroxyl interaction seems to be non-existent in case of HPMCAS, leading to an increase in pyroglutamate species with stress duration at 90° C. and 37° C. Also, the glass transition temperatures of AS-LF and AS-HF spray dried formulations with protein are slightly less than glass transition of trehalose spray dried protein. So, the mobility might be slightly higher in HPMCAS formulations restricting the hydrogen bonding interaction of the OH groups and N-terminal amine Fab groups. Without being bound by theory, there may be much more excess of HPMCAS excipients (at 1:1 w/w excipient to Fab loading) than trehalose thus the excess excipient that's not bound to protein might be driving instability and could be causing the glass transition temperature to be lower in case of spray dried HPMCAS powders.

The present data illustrates that when HPMCAS is added to an aqueous medium it could resist a change in pH round pH-5 (FIG. 7A). In neutral state (when, pH=7), it is soluble due to deprotonated state of its succinate groups (pKa-5), forming polymer colloids that are stable. The net negative charge on HPMCAS due to formation of succinate anion at neutral to basic pH prevents the polymer chains from interacting with each other. Upon increase in HPMCAS concentration, reaching maximum capacity to form colloids the polymer chains start to aggregate. With transitioning to a predominantly un-ionized protonated state of succinate group, gradually lowering the pH of the system (pH<4), and ultimately precipitating out. Such pH driven phenomenon of deprotonation and protonation of HPMCAS chains is reversible. Moreover, when pH is around pKa of succinate, HPMCAS/water mixture acts as a buffer absorbing any excess proton introduced in aqueous mixture. This buffering capacity was demonstrated in our titration studies with pure raw material and HPMCAS spray dried protein powder (FIGS. 7A and 7B). Taking advantage of this unique physicochemical property of HPMCAS, stable drug-polymer colloids (when ionized) and spray dried drug dispersions (when un-ionized) have been formulated (Friesen, D T et al., Mol Pharm, 2008, 5(6):1003-19). During addition of Fab2 stock at pH 5.5 for spray drying, HPMCAS/water mixture (pH˜6.5) didn't show a pH change. Suggesting, the hydrochloric acid protons from Fab2 solution being neutralized by succinate anions present in excipient, thus acting as a buffer.

Numerous publications have indicated that HPMCAS is able to maintain supersaturation for drugs in solution state due to the polymer's superiority as a precipitation inhibitor, forming a stable drug/polymer colloidal solution. Friesen et al. have reported occurrence of a colloidal state upon aqueous dissolution of spray dried drug dispersions made from HPMCAS that dissolve to form free drug and polymer chains upon dilution (Friesen, D T et al., Mol Pharm, 2008, 5(6):1003-19). That this colloidal state is amorphous in nature and important in maintaining drug solubility under physiological conditions. The present invention includes formation of a similar colloidal solution of HPMCAS and Fab2 upon exposure of HPMCAS spray dried Fab2 to water as well as during drug release from a PLGA rod (FIG. 3). While the pH of the rapidly forming colloids should be same as the pH of the pre-spray dried stock solution that was in a high energy colloidal state itself due to intermixing of HPMCAS stock (pH 6.5) and Fab2 stock (pH 5.5). This should be above the acid dissociation constant of the succinate group, causing the HPMCAS chains to ionize. Such a system consisting of ionized HPMCAS chains with Fab2, acting as electrostatic assemblies should be capable of neutralizing extrinsically added protons and act as a buffer at resisting pH changes. To demonstrate such properties of HPMCAS, the present examples shows released stable protein upon likely exposure to acidic protons from aqueous hydrolysis of a PLGA rod implant.

In preparation of protein loaded polymer rod the proper combination of excipients is necessary to withstand the high temperature processing condition of hot melt extrusion and acidic degradation products from PLGA hydrolysis (Rajagopal, K et al., J Pharm Sci, 2013, 102(8): 2655-66; Zhu, G and SP Schwendeman, Pharm Res, 2000, 17(3): 351-7). In general, the same excipients that were effective in maintaining the solid state stability as well as resisting pH change also facilitated Fab2 stability during in vitro release. Importantly, the present results indicate that HPMCAS excipients provide better protection against Fab2 aggregation within PLGA implant rods as compared to absence of any excipient or trehalose containing formulation. Similar results showing the effect of inorganic basic additives in protecting protein delivered from PLGA implants have been reported previously (Zhu, G and SP Schwendeman, Pharm Res, 2000, 17(3): 351-7). This further demonstrates that HPMCAS excipients may restrict non-covalent protein aggregation caused by the acidic microclimate emerging from PLGA hydrolysis products.

In other words, having a polymeric excipient with chemistry like acetate and succinate that could neutralize acidic degradants in a PLGA system aided in stable drug release. Possible interaction between the acidic hydrolysis products and Fab2 seem to diminish for a period of 3 weeks but the effect of long-term storage inside the rod environment will need to be studied in presence of HPMCAS.

### Example 2. Hydroxypropyl Methyl Cellulose Derivatives Stabilize Monoclonal Antibodies in Solid State and Resist pH Changes in Aqueous Phase

The Utility of acetate and succinate derivatives of hydroxypropyl methyl cellulose (HPMC or Hypromellose) in stabilizing three full-length monoclonal antibodies (MAbs) in the context of PLGA rods was evaluated. Two different HPMC derivatives were investigated; one with ˜8% acetate and ˜15% succinate (referred to as AS-LF) and the other with ˜12% acetate and 7% succinate acid (referred to as AS-HF). The materials and methods utilized for testing MAb formulations were as described in Example 1 for testing Fab2 formulations, with the exception of materials and methods specific to MAb formulations that are described below.

### Materials and Methods

Materials. MAb1, MAb2 and MAb3 were obtained from Genentech (South San Francisco, CA). MAb1 is a recombinant humanized IgG1K antibody reactive with HER2. MAb2 is a recombinant humanized IgG1_{κ} antibody reactive with VEGF-A. MAb3 is a recombinant humanized IgG1_{κ} antibody reactive with IgE.

Size Exclusion Chromatography (SEC). For MAb1, MAb2 and MAb3 Size-exclusion chromatography (SEC) HPLC was performed using an Agilent 1200 series HPLC system (Santa Clara, CA) equipped with a TOSOH TSKgel G3000SWXL (7.8×300 mm i.d., 5 μm particle size) column. Samples were analyzed at 25° C. in isocratic mode with 0.20 M K3PO4 and 0.25 M KCl, pH 6.2 as mobile phase at a flow rate of 0.5 mL/min. A 20 μL sample at 1 mg/mL concentration was injected, and the total run time was 30 min. Absorbance at 280 nm was used for detection. The percent peak area was calculated by dividing the peak area of each group at each time point to the total peak area.

Ion-Exchange Chromatography (IEC). For MAb1 Ion-exchange chromatography (IEC) HPLC was performed using an Agilent 1200 series HPLC system (Santa Clara, CA) on Thermo Scientific/Dionex (Sunnyvale, CA) ProPac WCX-10 analytical (4×250 mm) weak cation-exchange column and equipped with a diode array detector (DAD). Mobile phase A (solvent A) was 10 mM sodium phosphate buffer, pH 7.5, and mobile phase B (solvent B) was 100 mM sodium chloride dissolved in solvent A. Prior to analysis, the samples were diluted to approximately 1 mg/mL in solvent A, and 20 μL of sample was injected at a flow rate of 0.5 mL/min. A linear gradient starting from 85% solvent A at 0 min to 0% solvent A at 45 min was employed to separate MAb1 charge variants in a total of ˜60 min run time. Absorbance at 214 nm was used for detection. The IEC peaks were separated into a main peak, acidic peak, and basic peak.

For MAb2 Ion-exchange chromatography (IEC) HPLC was performed using an Agilent 1200 series HPLC system (Santa Clara, CA) on Thermo Scientific/Dionex (Sunnyvale, CA) ProPac WCX-10 analytical (4×250 mm) weak cation-exchange column and equipped with a diode array detector (DAD). Mobile phase A (solvent A) was 20 mM ACES buffer, pH 6.5, and mobile phase B (solvent B) was 200 mM sodium chloride dissolved in solvent A. Prior to analysis, the samples were diluted to approximately 1 mg/mL in solvent A, and 20 μL of sample was injected at a flow rate of 0.5 mL/min. A linear gradient starting from 70% solvent A at 0 min to 0% solvent A at 50 min was employed to separate MAb2 charge variants in a total of ˜90 min run time. Absorbance at 280 nm was used for detection. The IEC peaks were separated into a main peak, acidic peak, and basic peak.

For MAb3 Ion-exchange chromatography (IEC) HPLC was performed using an Agilent 1200 series HPLC system (Santa Clara, CA) on Thermo Scientific/Dionex (Sunnyvale, CA) ProPac WCX-10 analytical (4×250 mm) weak cation-exchange column and equipped with a diode array detector (DAD). Mobile phase A (solvent A) was 20 mM MES buffer, pH 6.1, and mobile phase B (solvent B) was 100 mM sodium chloride dissolved in solvent A. Prior to analysis, the samples were diluted to approximately 1 mg/mL in solvent A, and 20 μL of sample was injected at a flow rate of 0.8 mL/min. A linear gradient starting from 50% solvent A at 0 min to 0% solvent A at 60 min was employed to separate MAb3 charge variants in a total of ˜90 min run time. Absorbance at 280 nm was used for detection. The IEC peaks were separated into a main peak, acidic peak, and basic peak.

The performance of HPMCAS excipients in spray dried formulations with three full-length MAbs was compared to that of trehalose, metolose and a formulation devoid of excipients at 90° C. for a few hours and at 37° C. for a duration of several weeks. A temperature of 90° C. was selected to guide the compatibility of spray dried formulation for high temperature processing such as hot-melt extrusion used in the preparation of PLGA polymer rods for drug delivery. Stability at 37° C. was assessed to provide meaningful relative stability between the formulations that could be applicable to storage at 2-8° C. In addition, 37° C. also mimics body temperature and captures the effect of exposing the protein drug to physiological temperature experienced during long-term residence within a sustained delivery system comprising PLGA rods. Two of the three MAbs (MAb1 and MAb2), as a spray dried powder at E/P mass ratio of 1/1, were encapsulated within PLGA cylindrical rods by a hot melt extrusion method. Following which MAb stability inside PLGA rods when formulated in the presence of HPMCAS excipients and trehalose under physiological conditions was studied at 37° C. for a duration of several weeks. To characterize protein stability upon encapsulation in PLGA rods, MAb extracted from rods was examined for aggregation or chemical degradation. Although unlike trehalose which is hydrolyzable in humans, HPMCAS is non-biodegradable and hence applications in parenteral delivery are limited.

### Results

Spray Dried Formulations of MAbs. 13 formulations of each mAb at either 5 mg/mL or 10 mg/mL were prepared and evaluated for stability after spray drying. All of the 13 formulations were formulated in 10 mM His-HCl/His buffer and 0.01% w/v polysorbate 20. One of the formulations had only 10 mM His-HCl/His buffer and 0.01% w/v polysorbate 20 without any sugar or HPMCAS (control formulation). The formulations with HPMCAS excipients were formulated at pH 6.0±0.5, 10 mM His-HCl/His buffer and 0.01% w/v polysorbate 20. The preparations of spray dried AS-LF and AS-HF formulations with the MAbs is as shown schematically in FIG. 3 for Fab2. The mass ratio of trehalose, metolose, AS-LF and AS-HF excipient to protein in the respective formulations was 1:1, 1:4 and 4:1 (w/w). The molar ratio of excipient to protein however is different due to differences in the excipient molecular mass and ranged between 0.3 to 1752.9 depending on the formulation. The pH of all formulations were measured before spray drying in liquid state as well as after spray drying by reconstitution in water as shown in Table 2-1.for MAb1 and in Table 2-2 for MAb2. The pH measurements were similar indicating little change in ionization state due to spray drying.

Properties of Spray Dried MAbs. All 13 formulations after spray drying yielded micron-sized particles with similar particle morphology ranging from spherical to collapsed spheres. The particles containing MAb1 ranged from 3.8 to 10.0 microns in diameter across the different excipient formulations. The particles containing MAb2 ranged from 4.4 to 11.2 microns in diameter across the different excipient formulations. All spray dried powders were reconstituted in water and analyzed for aggregation using size-exclusion chromatography (SEC) and for chemical degradation using ion-exchange chromatography (IEC). For formulations with AS-LF and AS-HF, a separation protocol was followed before analyzing MAbs. The monomer content by SEC were similar for all formulations after spray drying and comparable to MAb formulations before spray drying (Table 2-1 and Table 2-2).

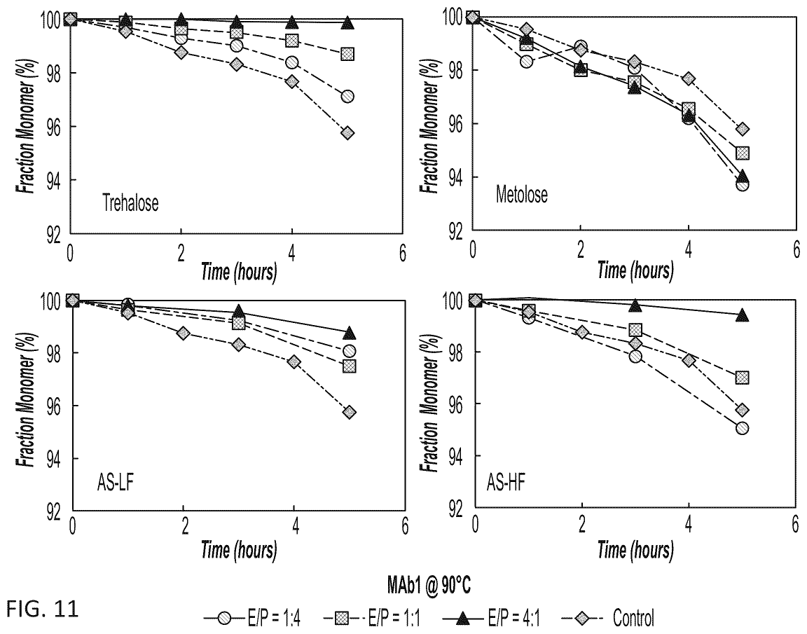

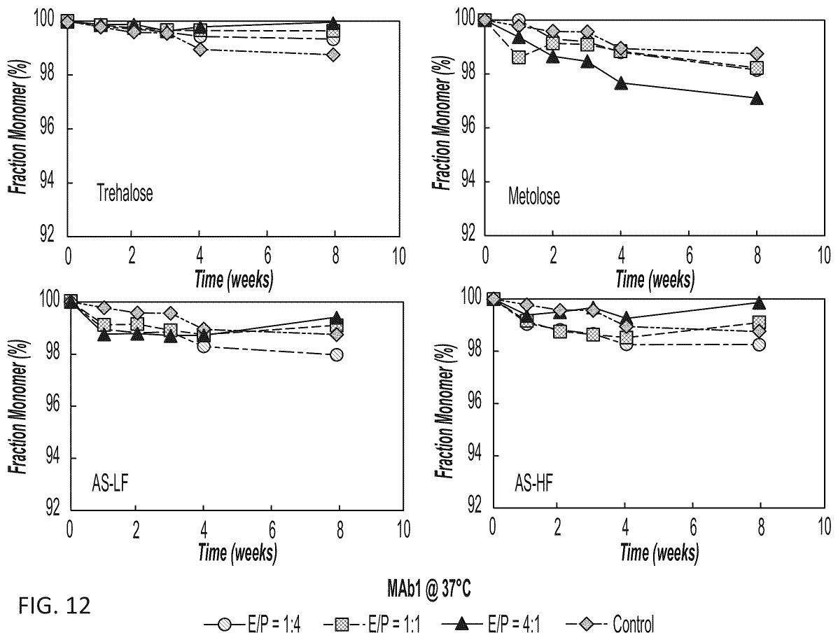

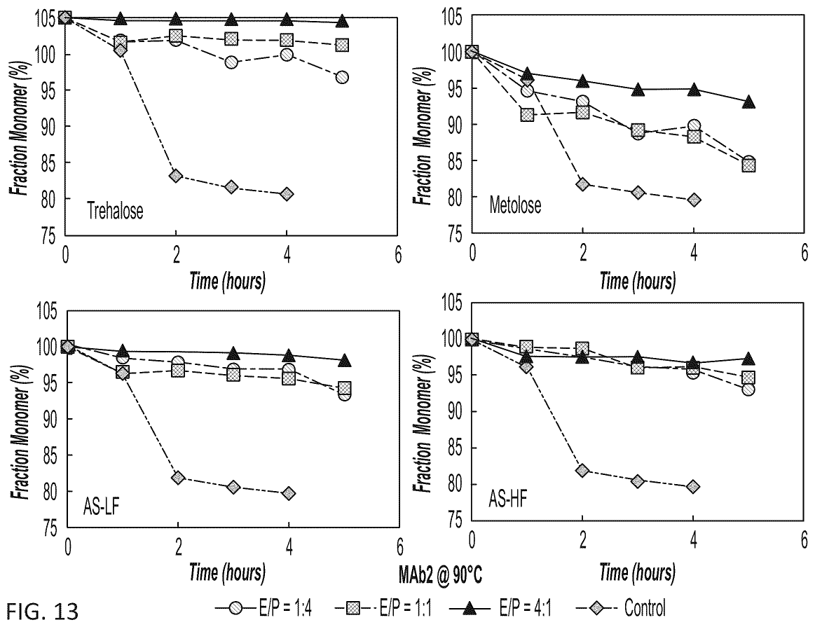

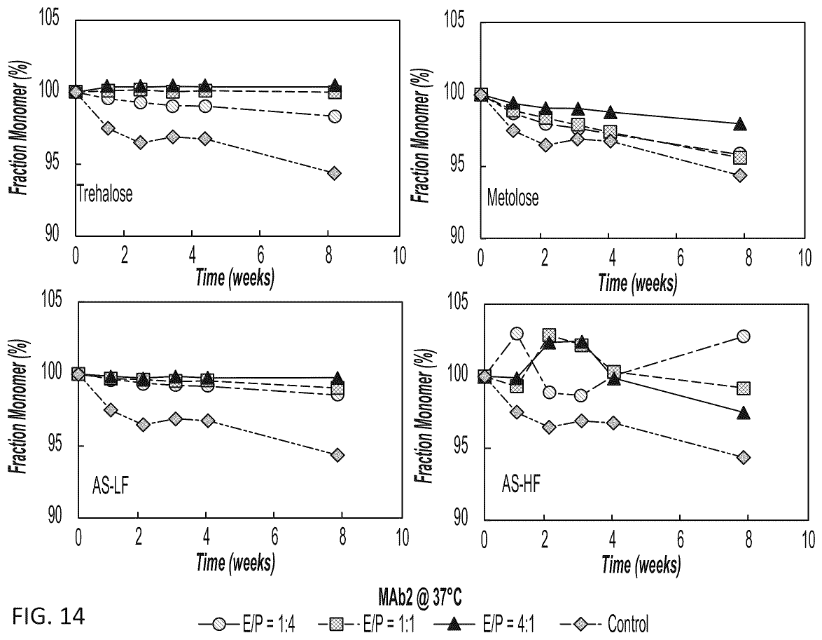

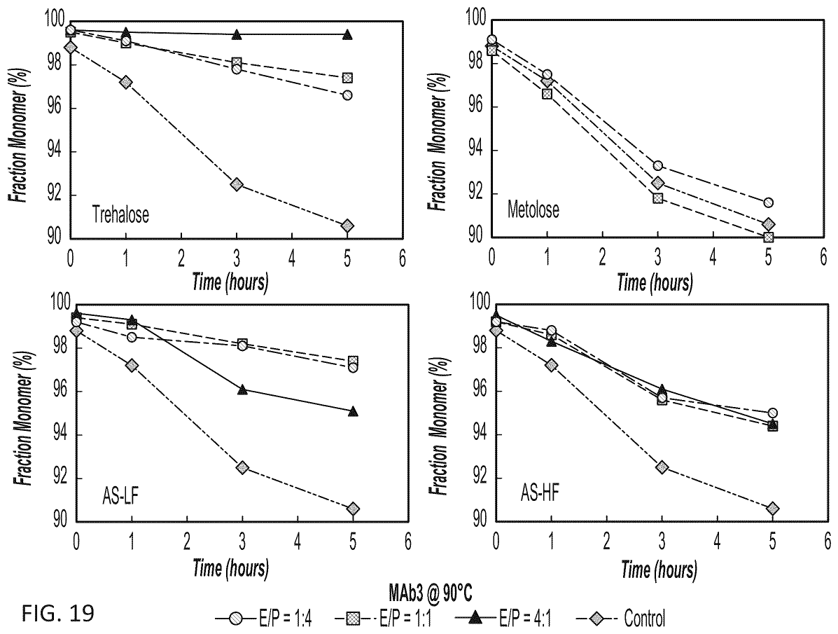

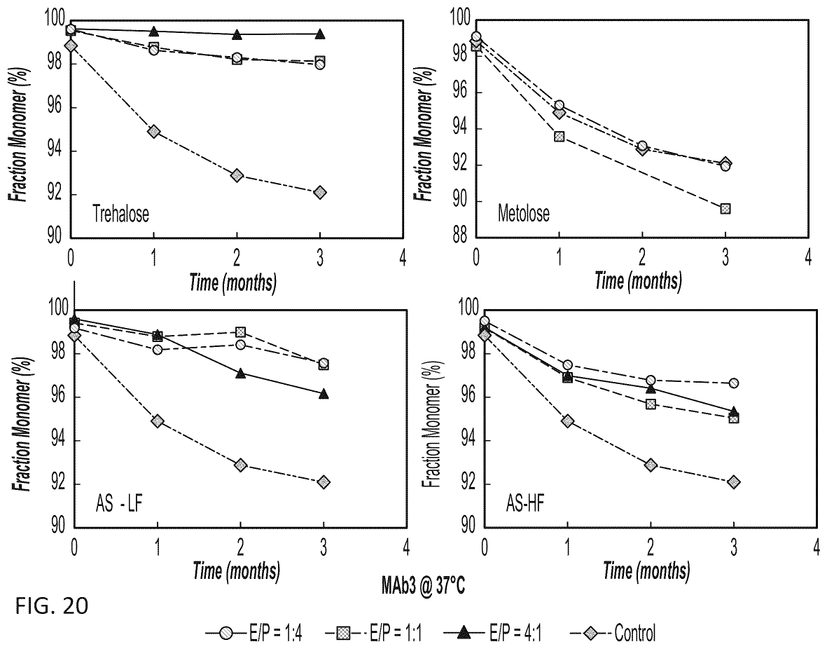

Effect of HPMCAS on Solid-State Stability of MAbs. The stability of MAbs in spray dried formulations was tested at 90° C. for several hours (see, FIG. 11, FIG. 13 and FIG. 19) and at 37° C. for several weeks (see, FIG. 12, FIG. 14 and FIG. 20). Maximum level of aggregation was observed in case of MAb formulations devoid of excipient (control) followed by MAb formulations containing metolose when stored at 90° C., and in the case of MAb3 when stored at 37° C.

TABLE 2-1
Details of spray dried MAb1 formulations evaluated for stability studies

| | | | | | Stability after | | | |
|---|---|---|---|---|---|---|---|---|
| | Excipient/ | | Spray dried | | spray drying | | | |
| | Protein | | powder | | | Main | | |
| | ratio | | Particle | | Monomer | Peak | pH | |
| Excipient | mg/ | mole/ | size | T_{g} | SEC | IEC | Before | After |
| (w/w)¹ | mg | mole | μm | ° C. | % | % | spraying | spraying² |
| E/P = 1/1 | | | | | | | | |
| Control | 0 | 0 | 6.6 | NA | 99.4 | 50.7 | 6.1 | 6.3 |
| Trehalose | 1 | 438 | 3.8 | 82.0 | 99.6 | 48.3 | 6.0 | 6.2 |
| Metolose | 1 | 1 | 5.3 | 150.2 | 98.8 | 50.4 | 6.0 | 5.8 |
| AS-LF | 1 | 1.6 | 9.0 | 123.7 | 99.7 | 47.3 | 6.5 | 6.5 |
| AS-HF | 1 | 1.1 | 9.4 | 109.3 | 99.7 | 46.2 | 6.5 | 6.7 |
| E/P = 1/4 | | | | | | | | |
| Control | 0 | 0 | 6.6 | NA | 99.4 | 50.7 | 6.1 | 6.3 |
| Trehalose | 0.25 | 110 | 6.7 | 129.7 | 99.5 | 49.5 | 6.0 | 6.2 |
| Metolose | 0.25 | 0.3 | 5.1 | 150.2 | 99.0 | 52.7 | 6.1 | 6.0 |
| AS-LF | 0.25 | 0.4 | 9.2 | 108.2 | 97.2 | 50.6 | 6.2 | 6.5 |
| AS-HF | 0.25 | 0.3 | 9.8 | 96.4 | 99.9 | 52.4 | 6.3 | 6.3 |
| E/P = 4/1 | | | | | | | | |
| Control | 0 | 0 | 6.6 | NA | 99.4 | 50.7 | 6.1 | 6.3 |
| Trehalose | 4 | 1752.9 | 7.1 | 81.7 | 99.8 | 54.5 | 6.0 | 6.0 |
| Metolose | 4 | 4.0 | 5.4 | 150.2 | 98.3 | 41.4 | 6.1 | 5.8 |
| AS-LF | 4 | 6.6 | 8.5 | 105.8 | 99.8 | 52.7 | 6.2 | 6.2 |
| AS-HF | 4 | 4.5 | 10.0 | 106.6 | 99.7 | 52.7 | 6.3 | 6.3 |

¹One sample of a the MAb1 formulation without an excipient (i.e., Control) was used for analyses of E/P = 1/1, E/P = 1/4 and E/P = 4/1 conditions.
²pH after spray drying was measured after dissolving 1% solid in water.

TABLE 2-2
Details of spray dried MAb2 formulations evaluated for stability studies

| | | | | | Stability after | | | |
|---|---|---|---|---|---|---|---|---|
| | Excipient/ | | Spray dried | | spray drying | | | |
| | Protein | | powder | | | Main | | |
| | ratio | | Particle | | Monomer | Peak | pH | |
| Excipient | mg/ | mole/ | size | T_{g} | SEC | IEC | Before | After |
| (w/w)¹ | mg | mole | μm | ° C. | % | % | spraying | spraying² |
| E/P = 1/1 | | | | | | | | |
| Control | 0 | 0 | 4.9 | NA | 95.8 | 58.5 | 6.2 | 6.1 |
| Trehalose | 1 | 438 | 7.0 | 78.4 | 94.0 | 60.0 | 6.0 | 6.3 |
| Metolose | 1 | 1 | 4.8 | 122.3 | 95.8 | 48.4 | 6.2 | 6.2 |
| AS-LF | 1 | 1.6 | 8.0 | 128.6 | 95.8 | 56.7 | 6.3 | 6.5 |
| AS-HF | 1 | 1.1 | 9.7 | 119.8 | 95.8 | 59.9 | 6.4 | 6.4 |
| E/P = 1/4 | | | | | | | | |
| Control | 0 | 0 | 4.9 | NA | 95.8 | 62.0 | 6.2 | 6.1 |
| Trehalose | 0.25 | 110 | 5.2 | 127.2 | 92.2 | 61.2 | 6.0 | 6.3 |
| Metolose | 0.25 | 0.3 | 4.0 | 120.2 | 95.8 | 54.1 | 6.0 | 6.0 |
| AS-LF | 0.25 | 0.4 | 7.7 | 130.8 | 95.8 | 57.4 | 6.5 | 6.5 |
| AS-HF | 0.25 | 0.3 | 8.6 | 123.2 | 95.8 | 60.0 | 6.2 | 6.1 |
| E/P = 4/1 | | | | | | | | |
| Control | 0 | 0 | 4.9 | NA | 95.1 | 76.9 | 6.2 | 6.1 |
| Trehalose | 4 | 1752.9 | 5.8 | 91.6 | 95.4 | 76.7 | 6.0 | 6.1 |
| Metolose | 4 | 4.0 | 4.4 | 150.2 | 99.7 | 72.9 | 6.0 | 5.9 |
| AS-LF | 4 | 6.6 | 7.5 | 65.9 | 98.2 | 74.5 | 6.1 | 6.0 |
| AS-HF | 4 | 4.5 | 11.2 | 106.6 | 95.8 | 59.9 | 6.3 | 6.3 |

¹One sample of the MAb2 formulation without an excipient (i.e. Control) was used in SEC and IEC analyses for E/P = 1/1 and E/P = 1/4 conditions, and a separate sample was used in SEC and IEC analyses for E/P = 4/1 condition.
²pH after spray drying was measured after dissolving 1% solid in water.

AS excipients and trehalose provided similar levels of protection to MAb1 against aggregation when stored at 90° C., with the greatest levels of protection observed in E/P=4 formulations. In contrast, metolose, which is devoid of acetate and succinate groups, did not protect MAb1 against aggregation.

In addition, AS excipients and trehalose provided similar levels of protection to MAb2 against aggregation when stored at 90° C., with the greatest levels of protection observed in E/P=4 formulations. In contrast, metolose, which is devoid of acetate and succinate groups, provided less protection to MAb2 against aggregation.

Moreover, AS excipients and trehalose provided similar levels of protection to MAb3 against aggregation when stored at 90° C., with the greatest levels of protection observed in E/P=¼ formulations. In contrast, metolose, which is devoid of acetate and succinate groups, provided little to no protection to MAb3 against aggregation.

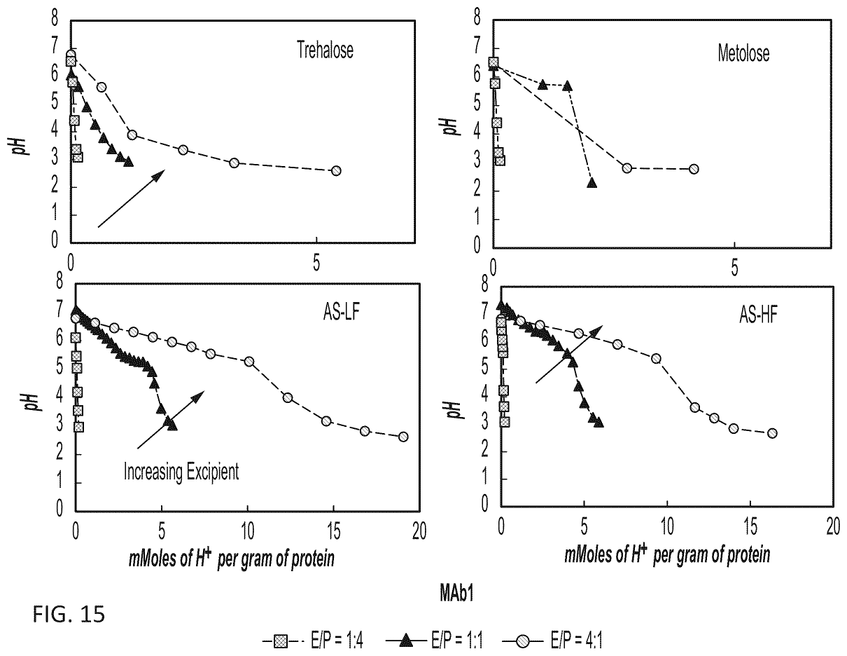

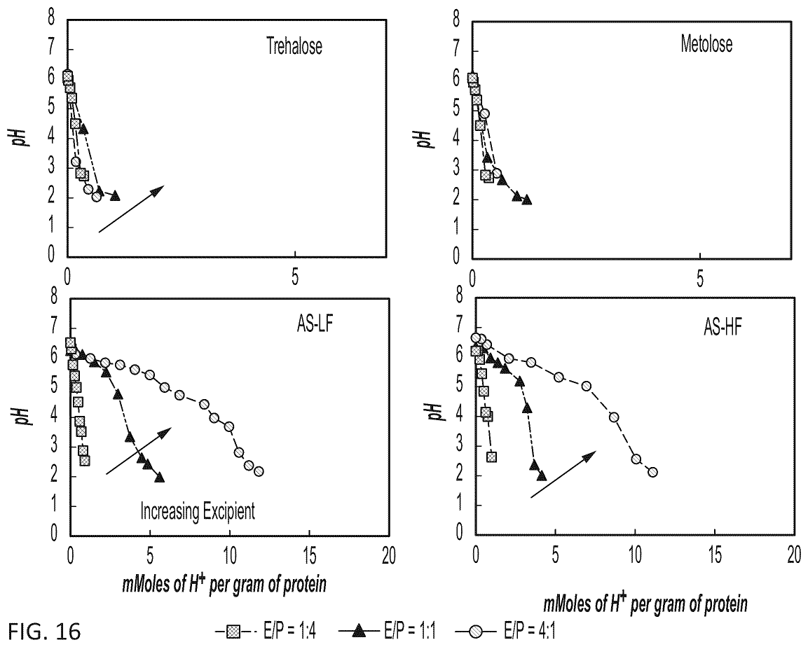

Buffering Capacity of HPMCAS Excipients. To examine the neutralization effect of HPMCAS excipients inside an acidic microclimate of a PLGA device, change in pH was monitored upon acid titration of HPMCAS spray dried powder formulations with strong hydrochloric acid (FIG. 15 and FIG. 16). The presence of HPMCAS excipient in the spray dried MAb powders resisted the pH change with addition of acid around pH 5 to pH 6. Upon comparison with spray dried protein powders without HPMCAS excipients the pH drop in case of trehalose and the control formulation lacking excipient significant (from pH 5.5-2.5) with addition of just 1 mM of acid. Similarly, in case of metolose which is structurally identical to HPMCAS minus the acetate and succinate functional groups, no buffering capacity was seen. Importantly, it was interesting to find that for HPMCAS containing spray dried protein formulations to affect a similar change in pH a much greater equivalent amount of the acid was required as compared to formulations without HPMCAS in formulations having a high E/P ratio.

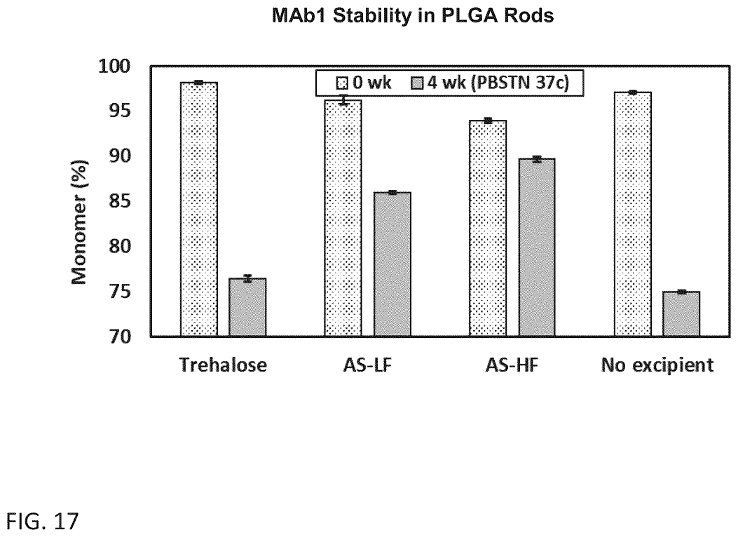

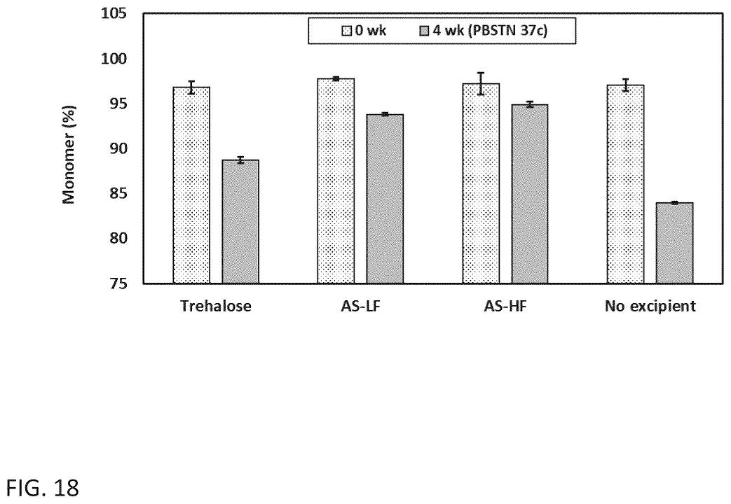

Stabilization of MAbs Encapsulated in PLGA Implant During In Vitro Release. To examine the effect of excipients on protein stability within a PLGA implant, spray dried MAb formulations with Trehalose, AS-LF, AS-HF and without any excipient were encapsulated within a 1 mg PLGA cylindrical rod for studying drug release and PLGA implant degradation. Recovered MAb from drug loaded PLGA rods stored at 37° C. for several weeks in PBSTN was analyzed for monomer stability (FIG. 17 and FIG. 18). Trehalose containing PLGA rods had a substantial loss in monomer content after 4 weeks. For both of the HPMCAS excipient formulations, much less MAb1 and MAb2 aggregates were observed.

1-19. (canceled)

20. A formulation comprising:
an antibody at a concentration of about 5 mg/mL to about 50 mg/mL,
a hydroxypropyl methylcellulose acetate succinate (HPMCAS), wherein the HPMCAS comprises about 8% acetate and about 15% succinate (HPMCAS-LF), and the ratio of the HPMCAS-LF to antibody in the formulation is about 4:1 (mg/mg), about 1:1 (mg/mg), or about 1:4 (mg/mg),
a histidine-HCl buffer at a concentration of about 5 mM to about 20 mM,
polysorbate 20 at a concentration of about 0.005% (w/v) to about 0.5% (w/v), and
wherein the pH of the formulation is about 5.5 to about 7.0.

21. The formulation of claim 20, wherein:
the antibody is present at a concentration of about 10 mg/mL,
the ratio of the HPMCAS-LF to the antibody in the formulation is about 4:1 (mg/mg), about 1:1 (mg/mg), or about 1:4 (mg/mg),
the histidine-HCl buffer is present at a concentration of about 10 mM,
the polysorbate 20 is present at a concentration of about 0.01% (w/v), and
wherein the pH of the formulation is about 6.5.

22. A formulation comprising:
an antibody at a concentration of about 5 mg/mL to about 50 mg/mL,
a hydroxypropyl methylcellulose acetate succinate (HPMCAS), wherein the HPMCAS comprises bout 12% acetate and about 7% succinate (HPMCAS-HF), and the ratio of the HPMCAS-HF to antibody in the formulation is about 4:1 (mg/mg), about 1:1 (mg/mg), or about 1:4 (mg/mg),
a histidine-HCl buffer at a concentration of about 5 mM to about 20 mM,
polysorbate 20 at a concentration of about 0.005% (w/v) to about 0.5% (w/v), and
wherein the pH of the formulation is about 5.5 to about 7.0.

23. The formulation of claim 22, wherein:
the antibody is present at a concentration of about 10 mg/mL,
the ratio of the HPMCAS-HF to the antibody in the formulation is about 4:1 (mg/mg), about 1:1 (mg/mg), or about 1:4 (mg/mg),
the histidine-HCl buffer is present at a concentration of about 10 mM,
the polysorbate 20 is present at a concentration of about 0.01% (w/v), and
wherein the pH of the formulation is about 6.5.

24. The formulation of claim 20, wherein the antibody is a monoclonal antibody.

25. The formulation of claim 24, wherein the antibody is a human antibody, a chimeric antibody or a humanized antibody.

26. The formulation of claim 25, wherein the antibody is an antibody fragment selected from a Fab, Fab′-SH, Fv, scFv, and (Fab′)2 fragment, optionally wherein the antibody fragment is a (Fab′)2 fragment.

27. The formulation of claim 24, wherein the formulation is spray dried to form a spray dried formulation of the antibody.

28. The spray dried formulation of claim 27, wherein the antibody in the formulation is stable at about 90° C. for at least about 5 hours and/or is stable at about 37° C. for at least about 4 weeks.

29. The spray dried formulation of claim 28, wherein the antibody in the formulation shows reduced aggregation and/or reduced chemical degradation.

30. The spray dried formulation of claim 29, wherein the reduced chemical degradation comprises reduced formation of succinimide variants of the antibody and/or reduced formation of pyroglutamate variants of the antibody.

31. The spray dried formulation of claim 27, wherein the formulation is an amorphous glassy formulation.

32. The spray dried formulation of claim 27, wherein the spray dried formulation of the antibody is encapsulated in a polymer system that produces acidic microclimate.

33. The spray dried formulation of claim 27, wherein the spray dried formulation of the antibody is encapsulated in lactic acid/glycolic acid polymer system.

34. The spray dried formulation of claim 27, wherein the spray dried formulation of the antibody is encapsulated in poly(lactic-co-glycolic acid) (PLGA).

35. The spray dried formulation of claim 34, wherein the spray dried formulation of the antibody is encapsulated in a PLGA rod.

36-42. (canceled)

43. A method of preparing a spray dried antibody, the method comprising preparing a formulation of claim 20, and subjecting the formulation to spray drying using a spray dryer comprising an inlet and an outlet.

44. (canceled)

45. The method of claim 43, wherein the inlet has a temperature of about 90° C. to about 120° C., and the outlet has a temperature of about 60° C.

46. (canceled)

47. A method of preparing a lactic acid/glycolic acid polymer particle comprising an antibody, the method comprising encapsulating the formulation of claim 20 in a lactic acid/glycolic acid polymer system.

48. A method of preparing a lactic acid/glycolic acid polymer particle comprising an antibody, the method comprising encapsulating the spray dried formulation of claim 27 in a lactic acid/glycolic acid polymer system.

49. A method of preparing a lactic acid/glycolic acid polymer particle comprising a polypeptide, the method comprising encapsulating a formulation comprising the polypeptide and a hydroxypropyl methylcellulose acetate succinate (HPMCAS) in a lactic acid/glycolic acid polymer system.

50-51. (canceled)

52. An article of manufacture comprising lactic acid/glycolic acid polymer particle encapsulated antibody prepared by the method of claim 47.

53. An article of manufacture comprising lactic acid/glycolic acid polymer particle encapsulated antibody prepared by the method of claim 48.
